(19) United States
(12) Patent Application Publication
(10) Pub. No.: US 2026/0193139 A1
(43) Pub. Date: Jul. 9, 2026

(54) STRETCHABLE AND HIGHLY CONDUCTIVE CRUMPLED GRAPHENE-BASED FILMS FOR MULTIFUNCTIONAL APPLICATIONS

(71) Applicant: The Penn State Research Foundation, University Park, PA (US)

(72) Inventors: Huanyu Cheng, University Park, PA (US); Jun Wang, Fuzhou (CN); Cheng Zhang, Fuzhou (CN)

(73) Assignee: The Penn State Research Foundation, University Park, PA (US)

(21) Appl. No.: 19/436,856

(22) Filed: Dec. 30, 2025

Related U.S. Application Data
(60) Provisional application No. 63/741,500, filed on Jan. 3, 2025.

(51) Int. Cl. C04B 35/628; A61B 5/01; A61B 5/11; C04B 35/52; C04B 35/78; H01G 11/10; H01G 11/36; H02N 1/04

(52) U.S. Cl. CPC C04B 35/62828; A61B 5/01; A61B 5/11; C04B 35/52; C04B 35/78; H01G 11/10; H01G 11/36; H02N 1/04; A61B 2560/0214; A61B 2562/0276; A61B 2562/125

(57) ABSTRACT

Embodiments relate to a nanocomposite material including a graphene-based 3D porous scaffold coated with active nanoparticles and to methods of making and using the material. The nanocomposite material exhibits high electrical conductivity, mechanical stretchability, and surface roughness, achieved through laser scribing, electrodeposition, and pre-strain transfer printing to induce crumpling. Embodiments further relate to a device including a crumpled nanocomposite material as an electrode and to methods of making and using the device.

### CROSS-REFERENCE TO RELATED APPLICATIONS

This application claims priority to U.S. Provisional Patent Application No. 63/741,500, which was filed on Jan. 3, 2025. The entirety of this application is incorporated by reference herein.

### STATEMENT REGARDING FEDERALLY SPONSORED RESEARCH DEVELOPMENT

This invention was made with government support under Grant Nos. 2319139, DGE2243979, ECCS2222654 and ECCS2309323 awarded by the National Science Foundation and under Grant No. EB030140 awarded by the National Institutes of Health. The Government has certain rights in the invention.

### FIELD

The present disclosure generally relates to a nanocomposite material including a graphene-based 3D porous scaffold coated with active nanoparticles and to methods of making and using the material. The present disclosure also relates to a device including a crumpled nanocomposite material as an electrode and to methods of making and using the device.

### BACKGROUND

The development of stretchable and flexible electronic devices has garnered significant interest in recent years, particularly within the fields of wearable, biomedical, and human-machine interface technologies. Advances in bio-integrated electrodes and soft electronics have driven the exploration of mechanically compliant materials capable of maintaining performance under various deformations. To meet these demands, researchers have focused on the fabrication of stretchable electronic components using a variety of nanomaterials, including metals, carbons, polymers, and metal oxides, which can be processed through techniques such as bulk micromachining, 3D printing, molding, and photonic sintering. Among these, laser-induced graphene (LIG) foams have emerged as promising candidates due to their three-dimensional porous structures, high surface area, and excellent electrical and thermal conductivity. These properties enable the creation of stable, stretchable devices such as sensors, energy storage systems, and soft actuators that can operate reliably during mechanical deformation.

The inherent porosity and defect sites within the LIG network can be further tailored through surface coatings or chemical functionalization, enhancing their chemical and structural properties. Hybrid nanocomposites combining LIG with other nanomaterials, such as metal or metal oxide nanoparticles, have been explored to improve electrochemical performance, mechanical strength, and electrical tunability.

Despite these advancements, challenges remain in scalable fabrication methods that can produce LIG-based nanomaterials with controlled morphology, composition, and physicochemical properties.

### SUMMARY

We have developed a scalable and cost-effective approach for the fabrication and patterning of nanocomposite materials with enhanced electrical and mechanical properties, suitable for use in wearable and bio-integrated electronic devices. In particular, our approach focuses on the development of crumpled, porous graphene-based nanocomposites integrated with active nanoparticles. These nanocomposites can be directly patterned into various geometries, enabling their integration into flexible, stretchable electronic systems for applications such as sensing, energy harvesting, storage, etc.

The fabrication method combines laser irradiation, electrodeposition, and a pre-strain strategy to produce crumpled graphene-based nanocomposites with a stable interface and tailored properties. The crumpled architecture enhances the material's stretchability and conformability, making it well-suited for wearable devices that must endure significant mechanical deformation while maintaining performance.

In exemplary embodiments, a method of forming a crumpled nanocomposite material includes forming a graphene-based three-dimensional, porous scaffold by laser scribing a substrate; depositing active nanoparticles onto the scaffold to form a nanocomposite material; transferring the nanocomposite material onto a pre-strained elastomeric substrate; releasing the pre-strain in the elastomeric substrate, thus forming the crumpled nanocomposite material; and optionally removing the elastomeric substrate from the crumpled nanocomposite material.

In some embodiments, the active nanoparticles include transition metal sulfides represented by the formula MₓS_{y}, wherein M is a transition metal, and wherein x and y are positive integers.

In some embodiments, the transition metal is nickel.

In some embodiments, the active nanoparticles include NiS₂.

In some embodiments, the active nanoparticles have a diameter between 10 nm and 1000 nm.

In some embodiments, the active nanoparticles have a diameter between 100 nm and 600 nm.

In some embodiments, the amount of pre-strain applied to the elastomeric substrate is from 1% to 200%.

In some embodiments, the method further includes, after depositing active nanoparticles onto the scaffold to form a nanocomposite material, annealing the nanocomposite material in an argon gas atmosphere.

In an exemplary embodiment, a system includes at least one sensing device including a crumpled nanocomposite material formed from the method described above, an electrolyte layer adjacent to the crumpled nanocomposite material, a first encapsulation layer, and a second encapsulation layer, wherein the crumpled nanocomposite material and the electrolyte layer are positioned between the first encapsulation layer and the second encapsulation layer; and an input/output device configured to receive data from the at least one sensing device.

In some embodiments, the system further includes a central computer device configured to receive data from the at least one sensing device and/or the input/output device.

In some embodiments the data includes one or more of motion data, temperature data, and toxic gas data.

In some embodiments, the at least one sensing device includes a first sensing device configured to collect motion data, a second sensing device configured to collect temperature data, and a third sensing device configured to collect toxic gas data.

In some embodiments, the sensing device is configured as a triboelectric nanogenerator.

In some embodiments, the sensing device is configured as a micro-supercapacitor array.

In some embodiments, the at least one sensing device includes a first sensing device configured as a triboelectric nanogenerator, a second sensing device configured as a micro-supercapacitor array, and a third sensing device configured as a biophysical sensor configured to collect one or more of motion data, temperature data, and toxic gas data.

In some embodiments, the first sensing device is configured to convert kinetic energy into electrical energy, the second sensing device is configured to store the electrical energy, wherein the electrical energy powers the third sensing device.

In an exemplary embodiment, a method for collecting sensor data includes providing a system including at least one sensing device including a crumpled nanocomposite material formed from the method described above, an electrolyte layer adjacent to the crumpled nanocomposite material, a first encapsulation layer, and a second encapsulation layer, wherein the crumpled nanocomposite material and the electrolyte layer are positioned between the first encapsulation layer and the second encapsulation layer; and an input/output device configured to receive data from the at least one sensing device; collecting sensor data via the at least one sensing device; and transmitting the sensor data to an input/output device for evaluation of the sensor data.

In some embodiments, the sensor data includes one or more of motion data, temperature data, and toxic gas data.

In some embodiments, the at least one sensing device includes a first sensing device configured as a triboelectric nanogenerator, a second sensing device configured as a micro-supercapacitor array, and a third sensing device configured as a biophysical sensor configured to collect one or more of motion data, temperature data, and toxic gas data.

In some embodiments, collecting sensor data includes converting kinetic energy into electrical energy via the first sensing device, storing the electrical energy via the second sensing device, powering the third sensing device via the electrical energy, and collecting the sensor data via the third sensing device.

Further features, aspects, objects, advantages, and possible applications of the present disclosure will become apparent from a study of the exemplary embodiments and examples described below, in combination with the Figures, and the appended claims.

### BRIEF DESCRIPTION OF THE DRAWINGS

The above and other objects, aspects, features, advantages and possible applications of the present innovation will be more apparent from the following more particular description thereof, presented in conjunction with the following drawings. Like reference numbers used in the drawings may identify like components.

FIG. 1 is a flow chart demonstrating an exemplary method of making a nanocomposite material.

FIG. 2 is an assembled view of an exemplary sensing device including the nanocomposite material as an electrode.

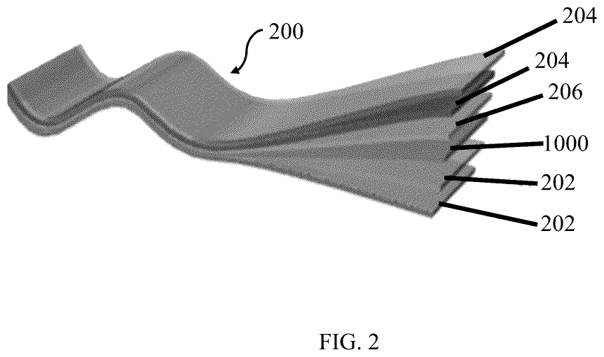

FIG. 3 is a schematic illustration of an exemplary method of making a nanocomposite material and sensing device.

FIG. 4A is a schematic block diagram illustrating an exemplary embodiment of a system for transmitting data collected by a device.

FIG. 4B is a schematic block diagram illustrating an exemplary embodiment of a system or platform for transmitting data collected by more than one device.

FIG. 4C is a schematic block diagram of an exemplary embodiment of a system or platform utilizing a plurality of devices for transmitting data to at least one input/output device for collection and evaluation of the data. The collected data can be evaluated by the input/output device or a central computer device that can be communicatively connected to the input/output device.

FIG. 5 is a schematic illustration of an exemplary use of a sensing device including the nanocomposite material.

FIG. 6 is a schematic illustration of a sensing device including the nanocomposite material.

FIG. 7 is a schematic illustration of a sensing device including the nanocomposite material.

FIG. 8 includes scanning electron microscopy images of (left) LIG foam, porous graphene/NiS₂ nanocomposite (left-center) before and (right-center) after transfer, followed by (right) the release of the pre-strain to yield crumped porous graphene/NiS₂ nanocomposite.

FIG. 9 includes (left) transmission electron microscopy (TEM) and (right) high-resolution TEM (HRTEM) of the crumpled porous graphene/NiS₂ nanocomposite.

FIG. 10 shows X-ray diffraction (XRD) patterns of LIG and crumpled porous graphene/NiS₂ nanocomposite, the inset showing the schematic crystal structure of NiS₂.

FIG. 11 shows a full survey X-ray photoelectron spectroscopy (XPS) spectra of crumpled porous graphene/NiS₂ nanocomposite.

FIG. 12 includes high-resolution (left) C Is, (center) S 2p, (right) Ni 2p XPS spectra of the crumpled porous graphene/NiS₂ nanocomposite.

FIG. 13 shows a Raman spectrum of LIG foam and crumpled porous graphene/NiS₂ nanocomposite.

FIG. 14 is a graph showing the linear relation of current-voltage (I-V) curves, suggesting the ohmic contacts between porous graphene foam and NiS₂ nanoparticles.

FIG. 15 is a graph showing the electromechanical properties of the nanocomposite with various εₚᵣₑ.

FIG. 16 is a graph showing the normalized resistance change (ΔR/R₀) of the porous graphene/NiS₂ nanocomposite with εₚᵣₑ of 200% as a function of the tensile strain from 0 to 200%. Error bars represent standard deviations over ten measurements.

FIG. 17 shows a photograph of the crumpled porous graphene/NiS₂ nanocomposite with εₚᵣₑ of 200% under an applied strain ranging from 0 to 200%.

FIG. 18 is a (left) graph showing nitrogen adsorption/desorption isotherms of porous graphene/NiS₂ nanocomposite with different εₚᵣₑ and the corresponding (right) Brunauer-Emmett-Teller (BET) surface area and the average pore size.

FIG. 19 is a graph showing the output voltage of the stretchable TENG (εₚᵣₑ: 0, 50, 100, 150, and 200%), with the photograph of the stretchable TENG (εₚᵣₑ of 200%, dimension of 5 cm×5 cm) shown in the inset.

FIG. 20 is a schematic showing the working principle of the crumpled porous graphene/NiS₂ nanocomposites-based TENG under cyclic compressive force. When the bottom and top electrodes are separated by the spacer at the initial state, there is no electrical output. Under a compressive force, electrostatic charges are generated and distributed on both the top and bottom electrodes. The gradual release of the compressive force allows the electrons to flow from the top to the bottom electrode via electrostatic induction. The device recovers to its original state when the compressive force is fully released, resulting in the maximum output voltage. Once the device is pressed again, electrons flow back until the device returns to the full-contact state.

FIG. 21 includes graphs showing output voltage and (c) current of the stretchable TENG (εₚᵣₑ of 200%) as the frequency increases from 1 to 5 Hz.

FIG. 22 is a graph showing output voltage of the stretchable TENG with increasing driving pressure.

FIG. 23 is a graph showing changes in the output voltage, current density, and power density with the increasing external load resistance.

FIG. 24 is a graph showing long-term stability of the stretchable TENG over 2,000 cycles of contact-separation motions before and after 3 months. Inset shows a magnified view of the voltage plot after three months.

FIG. 25 is a graph showing normalized output power density of the stretchable TENG as the applied biaxial and uniaxial tensile strain increases from 0 to 200% and followed by a full release. Error bars represent standard deviations over ten measurements.

FIG. 26 includes graphs showing the maximum output power density retention of the stretchable TENGs under different (left) uniaxial and (right) biaxial strains over 1000 cycles to show the long-term stability.

FIG. 27 is a graph showing output voltage of the stretchable TENG under different bending angles.

FIG. 28 is a graph showing charging and discharging curve of a commercial capacitor charged by the TENG driven by a linear mechanical motor through a commutator rectifier (discharge at 5 ηA), with the equivalent circuit diagram shown in the inset.

FIG. 29 is a schematic illustration showing the stretchable MSCA with an island-bridge layout.

FIG. 30 is a graph showing CV curves of the MSC at various scan rates.

FIG. 31 shows (left) cyclic voltammograms (CV) curves at various scan rates and (right) galvanostatic charge-discharge (GCD) plots at various current densities of the MSC cell.

FIG. 32 is a graph showing GCD curves of the MSC at various current densities.

FIG. 33 is a graph showing rate performance of the MSC cell with sequentially varied current density.

FIG. 34 is a graph showing specific capacitance of the MSC as a function of the scan rate (or current density as shown in the inset).

FIG. 35 is a graph showing GCD curves of the MSC cell at different temperatures (at a current density of 5 A g⁻¹).

FIG. 36 is a graph showing the specific capacitance values corresponding to FIG. 35.

FIG. 37 is a graph showing cycling stability and Coulombic efficiency of the MSC cell, with the corresponding electrochemical impedance spectroscopy (EIS) after 1 and 5,000 cycles shown in the inset.

FIG. 38 is Ragone plot of the gravimetric energy density versus gravimetric power density for the MSC from the Example in comparison with other MSCs previously reported in the literature.

FIG. 39 is a graph showing real-time collected CV curves of a single (dark) and the all-in-one MSCA (light) with serial connections.

FIG. 40 is a graph showing CV curves of MSCs connected in serial and/or parallel compared with a single MSC cell (at 10 mV s⁻¹).

FIG. 41 is a graph showing real-time collected CV curves of a single (dark) and the all-in-one MSCA (light) with parallel connections.

FIG. 42 shows (left) the equivalent circuit diagram of the four devices connected in parallel, and (right) relationship between the overall capacitance from the all-in-one MSCA and the number of devices connected in parallel.

FIG. 43 includes photographs of stretchable MSCAs (left) before and (right) after 50% biaxial stretching.

FIG. 44 includes graphs showing capacitance retention of the stretchable all-in-one MSCA comprised of four MSCs interconnected in series upon uniaxial/biaxial stretching from 0% to 200% (left) with and (right) without serpentine interconnects.

FIG. 45 includes graphs showing capacitance retention of the stretchable all-in-one MSCA with four MSCs connected in series upon (top) uniaxial/biaxial stretching from 0% to 200% and (bottom) bending at 180° over 1,000 cycles.

FIG. 46 includes graphs showing the relative resistance changes from bending-release movement of the (left) ankle, (center) elbow, and (right) knee.

FIG. 47 includes graphs showing the relative resistance changes from bending-release movement of the (left) fingers, and graphs showing the relative resistance changes from (center) swallowing, and (right) eye blinking.

FIG. 48 is a graph showing relative resistance changes from arterial pulse.

FIG. 49 includes (top-left) the stability test of the crumpled porous graphene/NiS₂-based piezoresistive pressure sensors over 55,000 cycles, with the dynamic response of the sensors (top-right) before and (bottom) after 55,000 cycles.

FIG. 50 is (left) a photograph of the as-prepared 6×6 sensor array to detect pressure distribution from (right) a heavy metal bar (top) and a lightweight plastic pipe (bottom), with the corresponding output shown in the right column.

FIG. 51 is a graph showing the real-time resistance changes of the temperature sensor with stepwise increased temperatures from 20 to 100° C., with the photograph of the sensor on the human skin shown in the inset.

FIG. 52 is a graph showing dynamic response of wearable temperature sensor upon contact and removal from the human body (room temperature of 28° C.).

FIG. 53 is a graph showing real-time sensing response of the gas sensor to NO₂ from 1 to 30 ppm, with the photograph of stretchable gas sensor on the wrist shown in the inset.

FIG. 54 is a graph showing the corresponding calibration curve with a linear relationship between the response and the NO₂ concentration. Error bars represent standard deviations over ten measurements.

FIG. 55 is a graph showing the corresponding calibration curve of a LIG-based gas sensor with a linear relationship between the response and the NO₂ concentration room temperature of 28° C. Error bars represent standard deviations over ten measurements.

FIG. 56 is a schematic showing the adsorption/desorption of gas molecules on the crumpled porous graphene/NiS₂ surface.

FIG. 57 is a graph showing the selectivity of the stretchable NO₂ gas sensor.

FIG. 58 is a (left) schematic and (b) circuit diagram of the integrated standalone platform on the human subject.

FIG. 59 is a graph showing the output voltage from the crumpled porous graphene/NiS₂-based TENG shoe sole driven by a human subject (body weight of 80 kg) at varying running frequencies.

FIG. 60 is a graph showing the real-time potential of the stretchable MSCAs with different numbers of MSCs connected in series/parallel, charged by the TENG driven by a human subject under different running frequencies.

FIG. 61 is a graph showing a comparison of the measured body temperature and pulse at wrist between the standalone stretchable device platform and the system powered by a commercial battery.

FIG. 62 includes graphs showing continuously monitored pulse wave signals over 6 hours (left) before and (right) after three months from the standalone stretchable device platform under the same indoor environmental conditions.

### DETAILED DESCRIPTION

The following description illustrates exemplary embodiments and methods of use that are presently contemplated for implementing the present invention. This description is not intended to be limiting, but rather to elucidate the general principles and features of various aspects of the invention. The scope of the invention is not restricted by this description.

Embodiments relate to a nanocomposite material including a graphene-based three-dimensional (3D) porous scaffold coated with active nanoparticles, as well as to methods of making and using the material. Embodiments further relate to devices, such as sensing devices and energy storage devices, including a crumpled nanocomposite material, and to methods of making and using the device.

Unless otherwise specified, all technical and scientific terms used herein have the meanings commonly understood by persons skilled in the art to which this invention pertains.

The term “crumpled” refers to a structural configuration characterized by a network of wrinkles, ripples, buckles, and/or the like formed within a material or composite, resulting from the application and subsequent release of mechanical pre-strain. This configuration imparts a three-dimensional, ripple-like morphology that increases the surface area of the material or composite.

### Nanocomposite Material

A nanocomposite material 1000 includes a graphene-based 3D porous scaffold coated with active nanoparticles. The scaffold features a porous, interconnected 3D network that serves as a conductive backbone for the deposition of the nanoparticles.

The graphene scaffold can be formed by laser scribing a suitable substrate to produce laser-induced graphene (LIG). The laser scribing parameters, such as wavelength, power, and scan speed, can be adjusted depending on the substrate material and desired electrical and mechanical properties of the material 1000. Suitable substrates include polyimide (PI), polyamide-imide (PAI), polyether sulfone (PES), polyphenylene sulfide (PPS), polyethylene terephthalate (PET), and polyethylene naphthalate (PEN). The resulting LIG scaffold possesses a porous, interconnected 3D network conducive to subsequent nanoparticle deposition.

The active nanoparticles enhance the electrochemical activity, electrical conductivity, and stability of the material 1000. It is understood that, due to the porous nature of the scaffold, the nanoparticles may not only coat the surface of the scaffold but may also be positioned throughout the depth of the scaffold.

The nanoparticles may include various transition metal sulfides represented by the formula MₓS_{y}, wherein M is a transition metal such as Fe, Co, Ni, Mo, W, Ta, Nb, or other metals capable of exhibiting electrochemical activity. The subscripts x and y are integers that define the stoichiometry and can be any positive integer.

In some embodiments, the transition metal sulfides are selected from nickel sulfides represented by the formula NiₓS_{y}, including NiS, NiS₂, Ni₃S₂, Ni₇S₆, Ni₉S₈, or other phases capable of electrochemical functionality. In particular embodiments, the active nanoparticles include NiS₂. However, x and y can be any positive integer

In embodiments where the active nanoparticles include nickel sulfides, the Ni:S ratio may be tuned to deliver certain properties to the material. A higher proportion of nickel ions may enhance electrical conductivity, whereas a higher proportion of sulfur ions may increase the number of active sites for pseudocapacitive redox reactions. Such as, the higher proportion of nickel ions of Ni—S binary compound, Ni₃S₂, NiS and Ni₉S₈ exhibit metallic properties, and the NiS₂ exhibits semiconductor properties. Meanwhile, the specific capacitance of NiS₂ is much larger than that of NiS, Ni₉S₈, Ni₃S₂.

The nanoparticles may be any suitable size. However, it is understood that the nanoparticles loaded on the substrate can serve as the cornerstone for achieving specific functionalities of materials, with the particle size directly influencing their physicochemical properties. For example, regarding a graphene/NiS₂ material, larger-sized NiS₂ nanoparticles, on the one hand, can increase the mass of the electrochemical energy storage material, which can help enhance the number of active sites for electrochemical energy storage, potentially improving the material's electrochemical storage capacity. On the other hand, due to the relatively low conductivity of semiconducting NiS₂ nanoparticles, larger-sized NiS₂ nanoparticles grown on the graphene substrate can adversely affect the conductivity of the composite material. This, in turn, may prevent some active materials from participating in electrochemical reactions, leading to a significant reduction in the electrochemical energy storage capacity per unit mass of the electrode material.

In some embodiments, the nanoparticles may have a diameter between 10 nm and 1000 nm, such as within 100 nm and 600 nm.

As non-limiting examples, the diameter of the nanoparticles may be at least 10 nm, at least 50 nm, at least 100 nm, at least 200 nm, at least 300 nm, at least 400 nm, at least 500 nm, at least 600 nm, at least 700 nm, at least 800 nm, at least 900 nm, and/or the like. As further non-limiting examples, the diameter of the nanoparticles may be no greater than 1000 nm, no greater than 900 nm, no greater than 800 nm, no greater than 700 nm, no greater than 600 nm, no greater than 500 nm, no greater than 400 nm, no greater than 300 nm, no greater than 200 nm, no greater than 100 nm, no greater than 50 nm, and/or the like.

The nanocomposite material 1000 can be transferred onto an elastomeric substrate to facilitate crumpling and mechanical deformation. Specifically, the elastomeric substrate may be pre-strained (pre-stretched) prior to printing or depositing the material 1000. After deposition, the pre-strain is released, causing the material 1000 to crumple into a ripple-like or wrinkled structure. This crumpled configuration enhances the stretchability of the material 1000, enabling conformal contact with various surfaces, such as human skin or flexible devices. The crumpling also increases surface roughness, which can improve sensing sensitivity and energy device performance. Even further, crumpling enables increased interfacial interactions between the material 1000 and charge carriers, thus improving electrical conductivity.

The extent of crumpling of the nanocomposite material 1000 can be controlled by the amount of pre-strain applied to the elastomeric substrate during the transfer process. Higher pre-strain levels generally result in a greater crumpled effect, while lower pre-strain levels produce a less pronounced crumpling.

The amount of pre-strain may be from about 1% to about 200%. As non-limiting examples, the pre-strain may be at least 1%, at least 5%, at least 10%, at least 25%, at least 50%, at least 75%, at least 100%, at least 125%, at least 150%, at least 175%, at least 190%, at least 195%, and/or the like. As further non-limiting examples, the pre-strain may be no greater than 200%, no greater than 195%, no greater than 190%, no greater than 175%, no greater than 150%, no greater than 125%, no greater than 100%, no greater than 75%, no greater than 50%, no greater than 25%, no greater than 10%, no greater than 5%, and/or the like.

In some embodiments, the elastomeric substrate may be removed either before or during the integration of the nanocomposite material 1000 into a device 200. In other embodiments, the elastomeric substrate remains as part of the final device structure, providing mechanical support and flexibility.

An example method 100 for forming the nanocomposite material 1000 may include the following steps:

Step 102—Formation of the graphene scaffold by laser scribing a suitable substrate, such as PI, PAI, PES, PPS, PET, or PEN. Laser parameters, such as wavelength, power, and scan speed, are selected to optimize the electrical and mechanical properties of the resulting scaffold.

Step 104—Deposition of active nanoparticles onto the graphene scaffold using suitable techniques such as electrodeposition. In some embodiments, the active nanoparticles form a uniform or substantially uniform coating. In preferred embodiments, the active nanoparticles are deposited onto the graphene scaffold without use of a polymeric binder and/or a conductive additive. Optionally, the method may include a step of annealing the nanocomposite material in argon.

Step 106—Transfer of the coated scaffold onto a pre-strained elastomeric substrate via transfer printing or similar techniques.

Step 108—Release of the pre-strain in the elastomeric substrate, inducing crumpling of the nanocomposite material into a ripple-like or wrinkled morphology.

(Optional) Step 110—Removal of the elastomeric substrate from the crumpled nanocomposite material, if desired, for subsequent device fabrication or application.

### Device

The crumpled nanocomposite material 1000 may be integrated into a device 200, such as a sensing device or energy storage device. In particular, the nanocomposite material 1000 may serve as an electrode within the device.

The device 200 may include additional layers configured to encapsulate the nanocomposite material 1000, provide mechanical support, and/or facilitate interaction between the nanocomposite material 1000 and an underlying substrate.

The nanocomposite material 1000 has a first surface and an opposite second surface. In some embodiments, one or more layers 202 may be positioned directly or indirectly adjacent to the first surface of the material 1000. The device may include one, two, three, or more layers 202 arranged on or near the first surface of the material 1000.

In other embodiments, one or more layers 204 may be positioned directly or indirectly adjacent to the second surface of the material 1000. The device may include one, two, three, or more layers 204 arranged on or near the second surface of the material 1000.

In some embodiments, one or more layers 202 may be positioned directly or indirectly adjacent to the first surface of the material 1000 and one or more layers 204 may be positioned directly or indirectly adjacent to the second surface of the material 1000.

The layers 202 and 204 may be composed of any suitable material. In some embodiments, these layers are formed from flexible and/or stretchable polymers. Certain embodiments may use hydrophilic polymers. Examples include, but are not limited to, polydimethylsiloxane (PDMS), polyurethane (PU), thermoplastic polyurethane (TPU), commercially available silicones such as Ecoflex, polyimide (PI), poly(lactic-co-glycolic acid) (PLGA), parylene, and other materials with comparable chemical or functional properties. These materials are selected for their mechanical flexibility, biocompatibility, and durability.

The device 200 may further include an electrolyte 206, which facilitates electrical conduction within the device. The electrolyte 206 may be positioned such that it is in direct contact with the nanocomposite material 1000, enabling charge transfer.

In some embodiments, the electrolyte 206 may be a gel or solid electrolyte. In such cases, the electrolyte 206 may be formed as a layer that directly overlays or underlies the nanocomposite material 1000. Alternatively, the electrolyte 206 may be an aqueous solution, such as a liquid electrolyte, that contacts the nanocomposite material 1000 directly.

In one particular embodiment, the device 200 may include, in sequence, one or more layers 204 on one side, an electrolyte 206, the nanocomposite material 1000, and one or more layers 202 on the opposite side.

The device 200 can be configured to collect and transmit sensor data related to various physical and environmental parameters. The data may include metrics such as human motion and gestures, which encompass motion trajectories, position, orientation, rotation, and other kinematic parameters. The sensor data can also include temperature measurements, such as body temperature, and gas detection data for environmental monitoring, including concentrations of toxic or hazardous gases.

For example, in wearable applications, the device 200 can monitor human motion, enabling activity tracking, gait analysis, gesture recognition, etc. Such data are valuable for robotics, human-machine interface (HMI) systems, health monitoring, rehabilitation, prosthetics, personalized healthcare/fitness tracking, virtual reality, etc. The device's high sensitivity to mechanical deformation allows it to detect subtle motions, including joint flexion, muscle movement, and even fine finger movements.

The device 200 can also be configured to measure temperature, such as body temperature, for health monitoring applications. Temperature sensors based on the nanocomposite material 200 can detect variations in thermal energy transfer, providing continuous or intermittent temperature readings, which can be useful in medical diagnostics, fever detection, or environmental temperature monitoring.

Additionally, the device 200 can function as a gas sensor, capable of detecting specific gases such as nitrogen dioxide (NO₂), ammonia (NH₃), carbon monoxide (CO), and others. The nanocomposite material 1000's surface properties may facilitate gas adsorption and charge transfer, enabling sensitive detection of trace gases in the environment. This application is relevant for environmental safety, industrial monitoring, and hazardous gas detection in confined or outdoor settings.

The device 200 can be configured as a triboelectric nanogenerator (TENG). TENGs operate on the principle of contact electrification and electrostatic induction. For example, two materials with different triboelectric properties come into contact and then separate; they generate a voltage difference, producing an electrical current. In wearable devices, the TENG can harvest mechanical energy generated from human motion, such as walking, running, or joint movement, and convert it into electrical energy. This energy can be stored or directly used to power sensors or wireless modules. In the context of the present disclosure, the crumpled nanocomposite material 1000, with its high surface roughness and flexibility, may enhance contact area and charge transfer efficiency, resulting in higher output voltage and current.

The device 200 may also be configured as a micro-supercapacitor array (MSCA). MSCAs are high-performance energy storage devices consisting of interdigitated electrodes separated by a suitable electrolyte. They provide rapid charge/discharge cycles and high power density, making them suitable for powering wearable electronics and sensor systems. The nanocomposite material 1000, with its high surface area and electrical conductivity, can serve as an electrode material for MSCAs.

Embodiments can include one or multiple device 200 units. For example, a single system may include multiple devices operating independently or collectively. Each device can function without interference, enabling distributed sensing or energy harvesting across different body parts or environmental zones. The number of devices can range from one to many, including configurations with two, three, four, or more units.

In other embodiments, multiple devices 200 can operate as a unified platform. For instance, one device may function as a TENG to harvest mechanical energy; another as an MSCA to store the harvested energy; and additional devices may serve as sensors for temperature, gas detection, or motion monitoring. The integrated system can convert kinetic energy from human motion into electrical energy, store it, and power sensitive biophysical sensors for continuous health monitoring, environmental sensing, or human-machine interaction.

In one particular example of a unified platform, human motion may cause the TENG to generate electrical energy, which can be stored in the MSCAs. The stored energy can then power sensors and wireless modules that transmit data in real time. This configuration enables a self-powered, wearable sensing platform capable of long-term operation without external power sources.

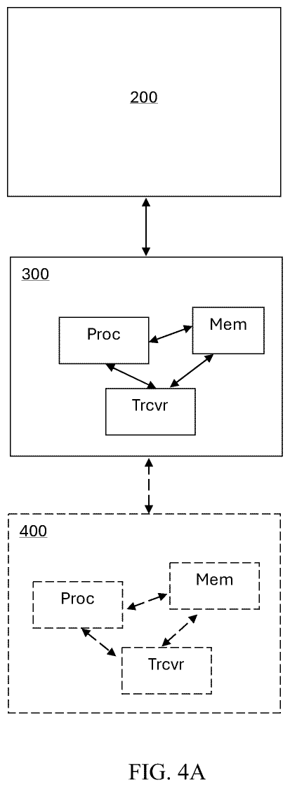

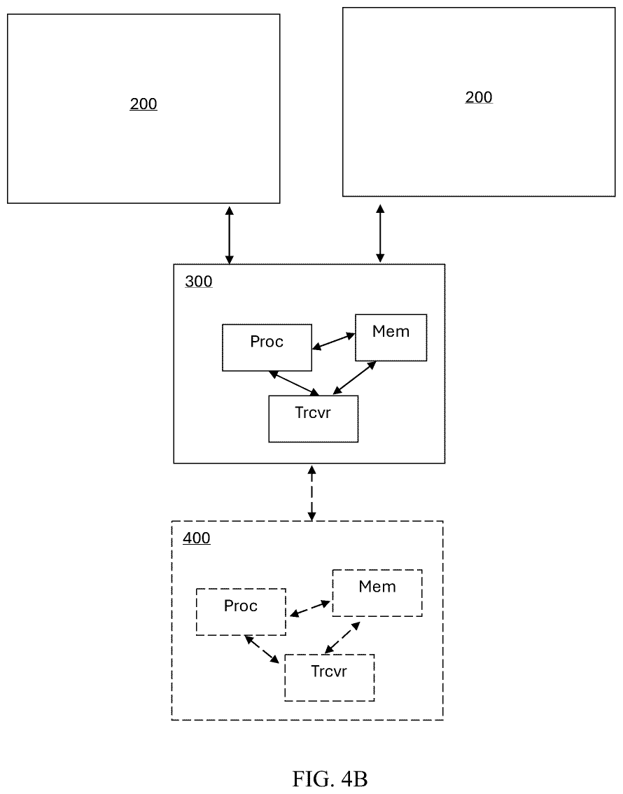

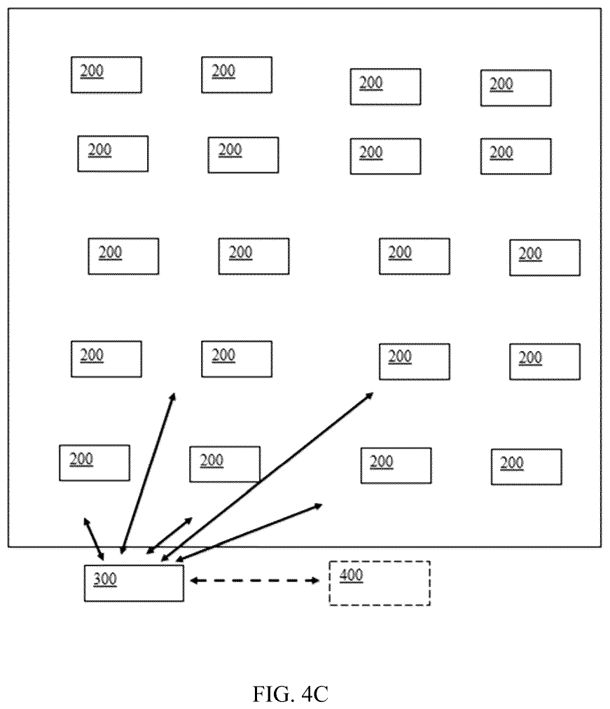

Referring to FIGS. 4A, 4B, and 4C, the device 200 can be hardwire connected to an input/output device 300 (e.g., a smart phone, tablet, laptop computer, personal computer, computer device of a drone or robotic device, etc.), or the device 200 can be communicatively connected to the input/output device 300 via a network connection or wireless connection (e.g., internet connection, wide area network connection, near field communication connection, Bluetooth connection, etc.).

In some embodiments, the input/output device 300 can be configured to receive data from the device 200 for storage and analysis. In some implementations, the input/output device 300 can be configured as a server or cloud-based service providing device for storage and analysis of the data obtained via the device 200. The data can be communicated to a user via display device, which can be a tablet, smartphone, laptop computer, personal computer, or other type of terminal device. The display device can be effectuated via an application programming interface (API) and/or use of an application stored on the display device. It is contemplated that the input/output device 300 can include the display device, or the display device can be a separate device.

In some embodiments, the device 200 can alternatively or subsequently be sent to a central computer device 400 (e.g., a server, an operator workstation, etc.) that can be hardwire connected to the device 200 and/or the input/output device 300, or can be communicatively connected to the device 200 and/or the input/output device 300 via a network connection and/or a wireless connection. The central computer device 400 can be configured to store, analyze, and/or display data received from the device 200 and/or input/output device 300.

In some embodiments, the collected data can be continuously streamed to the input/output device 300 and/or the central computer device 400. In other embodiments, the collected data can be periodically streamed to the input/output device 300 and/or the central computer device 400 (e.g., non-continuously at pre-determined intervals). Embodiments of the device 200 can be configured to provide real time data collection.

The input/output device 300 and/or the central computer device 400 can be a computer device that can include a processor (Proc.) connected to a non-transitory memory (Mem.) and at least one transceiver (Trcvr) for forming communicative connections with one or more other devices. The at least one transceiver (Trcvr) can include a Bluetooth module and/or other type of transceiver unit (Trcvr). The processor can be hardware (e.g., processor, integrated circuit, central processing unit, microprocessor, core processor, computer device, etc.), configured to perform operations by execution of instructions embodied in algorithms, data processing program logic, artificial intelligence programming, automated reasoning programming, etc., that can be defined by code stored in the memory. The processor can facilitate receipt, processing, and/or storage of readings from the device 200 and/or control transmission of the collected data to input/output device 300 and/or the central computer device 400.

It should be noted that use of processors herein can include hardware, such as for example any one or combination of a Graphics Processing Unit (GPU), a Field Programmable Gate Array (FPGA), a Central Processing Unit (CPU), a microprocessor, a processor, etc. The processor can include one or more processing or operating modules. A processing or operating module can be a software or firmware operating module configured to implement any of the functions disclosed herein. The processing or operating module can be embodied as software and stored in non-transitory memory, the memory being operatively associated with the processor. A processing module can be embodied running a web application, a desktop application, a console application, etc.

The memory (Mem.) can be a non-transitory computer readable memory configured to store data. Embodiments of the memory can include a processor module and other circuitry to allow for the transfer of data to and from the memory, which can include to and from other components of a communication system. This transfer can be via hardwired links or wireless transmission communication links. The communication system can include transceivers, which can be used in combination with switches, receivers, transmitters, routers, gateways, waveguides, etc., to facilitate communications between different devices via a communication approach or protocol for controlled and coordinated signal transmission and processing to any other component or combination of components of the communication system. The transmission can be via a communication link, which can be a wireless type of communication connection and/or a wired type of connection.

The computer or non-transitory machine-readable medium can be configured to store one or more instructions thereon. The instructions can be in the form of algorithms, program logic, etc., that cause the processor to execute any of the functions disclosed herein.

The processor can be in communication with other processors of other devices (e.g., additional external device, a computer system, a laptop computer, a desktop computer, etc.). An exemplary other device can be a Bluetooth enabled device, near field communication device, etc. Any of those other devices can include any of the exemplary processors disclosed herein, as well as transceivers or other communication devices/circuitry to facilitate transmission and reception of wireless signals or other types of communicative connections.

Either the input/output device 300 and/or the central computer device 400 can be configured to be connected to other input devices and output devices. Examples of input devices can include a scanner device (e.g., scanner), a microphone, a keyboard, a touch screen, a button, a sensor, a detector, or other type of input device. Examples of output devices can include a display, a printer, a speaker, or other type of output device.

As noted above, once collected data is transmitted to the input/output device 300 and/or the central computer device 400, the data can be analyzed and evaluated to determine an output. For example, the data can be processed and evaluated to determine motion or gestures, temperatures, presence or concentration of gas, etc. In some embodiments, the data can be processed using artificial intelligence or machine learning algorithms stored on the input/output device 300 and/or the server central computer device 400. In particular, the input/output device 300 and/or the server central computer device 400 can run a program that uses the collected data along with a module trained via a machine learning process that received the collected sensor data and processes that data to determine an output.

### EXAMPLES

### Materials

The chemicals used for this Example were of analytical reagent grade. Polyimide (PI) film, NiCl₂·6H₂O, CH₄N₂S, KOH, Nafion, PVA (molecular weight: 75 000-80000 g/mol), and polydimethylsiloxane (PDMS) were purchased from Aladdin Chemistry Co., Ltd. (Shanghai, China). Solutions were freshly prepared with deionized water.

### Fabrication of Crumpled Porous Graphene/NiS₂ Nanocomposites

After laminating a cleaned PI film on a spin-coated thin elastomer substrate (PDMS), laser scribing of the PI with a computer-controlled CO₂ laser (10.6 μm with pulse duration of ~14 μs, laser power of 3.8 W) under ambient conditions prepared the porous graphene foam. NiS₂ nanoparticles were electrodeposited on the LIG scaffold (2.0×3.0 cm²) as the working electrode in a three-electrode electrochemical system, where a Pt sheet (1.0×1.0×0.1 cm³) and a saturated Ag/AgCl were used as the counter and reference electrodes, respectively. An aqueous solution with 25 mM NiCl₂·6H₂O and 1 M CH₄N₂S was prepared as the electrolyte. NiS₂ nanoparticles were electrodeposited by cyclic voltammetry (CV) with the potential range from −1.4 to 0.1 V at a scan rate of 3 mV s⁻¹ for 1,000 s. Next, the prepared samples were washed thoroughly with deionized water and annealed at 220° C. in an argon gas atmosphere for 1 h with a heating rate of 5° C. min⁻¹. Carefully laminating the LIG/NiS₂ film supported by the Si chip substrate to a pre-stretched acrylic elastomer film (3M VHB 4905), followed by peeling off the Si chip and releasing the pre-strain, formed crumpled structure in the LIG/NiS₂ film on VHB. PDMS film was spin-coated on the crumpled LIG/NiS₂ film, and then the crumpled LIG/NiS₂ film supported by PDMS film was obtained after peeling off the VHB film by heating at 120° C. for 1 h.

### Fabrication of Crumpled Porous Graphene/NiS₂-Based TENG

Two PDMS films with crumpled porous graphene/NiS₂ nanocomposites as the top and bottom electrodes were separated by four PDMS cylinders as the spacer, resulting in crumpled porous graphene/NiS₂-based stretchable TENG (length of 5 cm and width of 5 cm).

### Fabrication of Crumpled Porous Graphene/NiS₂-Based MACAs

First, the PVA/KOH gel electrolyte was prepared by adding 3.58 g of KOH into 60 mL of deionized water, followed by adding 6 g of PVA powder, which was heated to 95° C. under stirring until the solution became clear. Next, the PVA/KOH gel electrolyte was drop-coated on top of the crumpled porous graphene/NiS₂ interdigitated electrodes on the PDMS substrate using a syringe. After 10 min to fully diffuse the electrolyte into the porous structure, the sample was further heated to and kept at 35° C. for 12 h to remove excess water in the electrolyte. Laminating a cleaned PDMS film on top of the gel electrolyte as an encapsulation layer yielded the crumpled porous graphene/NiS₂ nanocomposites-based MSCAs.

### Fabrication of Crumpled Porous Graphene/NiS₂-Based Biophysical Sensor

The crumpled porous graphene/NiS₂-based piezoresistive pressure/temperature sensors were obtained by connecting a serpentine/rectangular crumped porous graphene/NiS₂ electrode with two Cu lead wires on the PDMS substrates, followed by packaging with a thin PDMS layer. The crumpled porous graphene/NiS₂-based gas sensor was obtained by connecting needlelike crumpled porous graphene/NiS₂ gas-sensitive region with two Cu lead wires on the PDMS substrates.

### Characterization

The morphology of samples was characterized by an optical camera (D750, Nikon, Japan), scanning electron microscopy (SEM, Hi-tachiS3400, Japan), and transmission electron microscopy (TEM, JEOL-2100F, Japan). The structural and electronic properties of samples were characterized by X-ray diffraction patterns (XRD, Thermo ARL X'TRA, Switzerland), X-ray photoelectron spectroscopy (XPS, Ulvac-Phi, Inc., Japan), and Raman spectra (HORIBA, LabRAM HR 800, France). Electrochemical performance measurements were performed on an electrochemical workstation (CHI 660D, Chenhua Instruments, China). The voltage, current, and resistance were measured using a multimeter (DMM 7510, Keithley, Cleveland, OH, USA).

### Gas-Sensing Testing

Before the start of the gas-sensing experiment, dry air was passed into the test chamber for one hour to ensure stable resistance. Next, the test gas (including NO₂, NO, CO₂, NH₃, CO, acetone, SO₂, Methane, H₂S, and Ethanol) was delivered by controlling a dynamic gas distribution instrument (Tanggao Electric Technology, GC400, China) with a constant flow of 200 sccm. The resistances of gas sensors were captured using a multimeter system (DMM 7510, Keithley, Cleveland, OH, USA).

### Electrochemical Calculation

The capacitance C of electrode materials and devices was calculated from the GCD curves using the following equation:

$$C = ( I \times Δ t ) / Δ V ,$$

where I, Δt, and ΔV are the discharge current, time, and potential range, respectively. The gravimetric (or areal) specific capacitance C_{g} (or C_{A}) was calculated as the ratio of the capacitance to the mass of the active material (or area of the electrode/device). The energy and power densities based on the mass of active materials were then evaluated from GCD curves using the following equations:

$${E}_{g} = \frac{1}{2} \times {C}_{g} \times \frac{{( Δ V )}^{2}}{3600} , {E}_{A} = \frac{1}{2} \times {C}_{A} \times \frac{{( Δ V )}^{2}}{3600}$$

$${P}_{g} = \frac{{E}_{g}}{Δ t} \times 3600 , {P}_{A} = \frac{{E}_{A}}{Δ t} \times 3600$$

### Results and Discussion

### Fabrication of the Patterning Crumpled Porous Graphene NiS₂ Nanocomposite Films

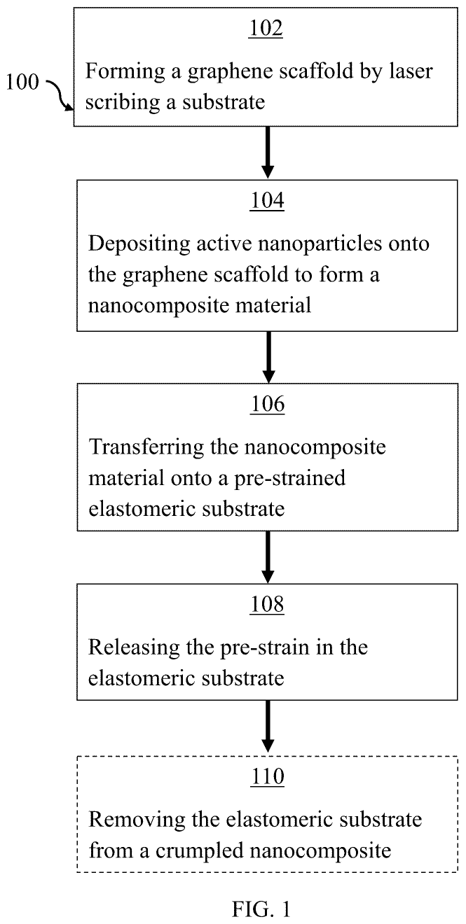

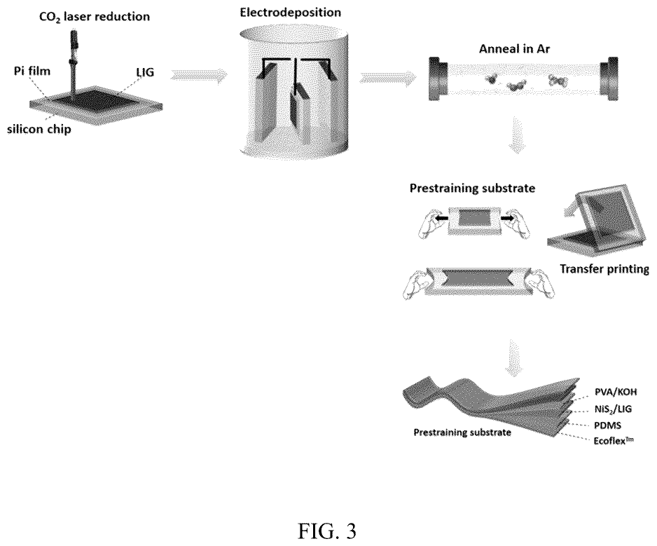

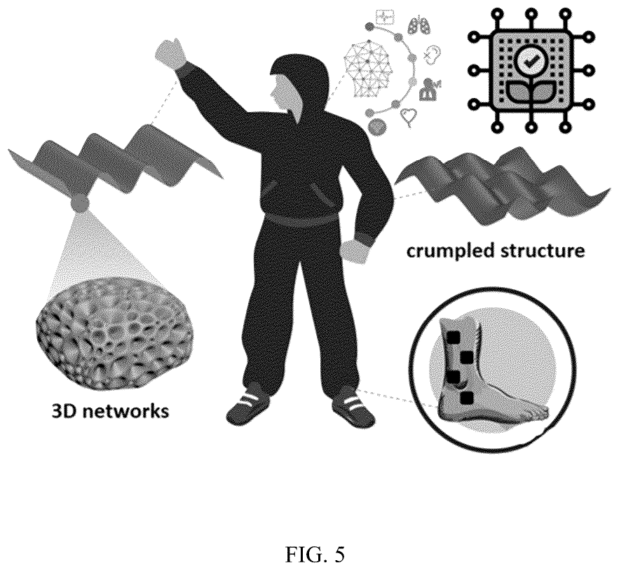

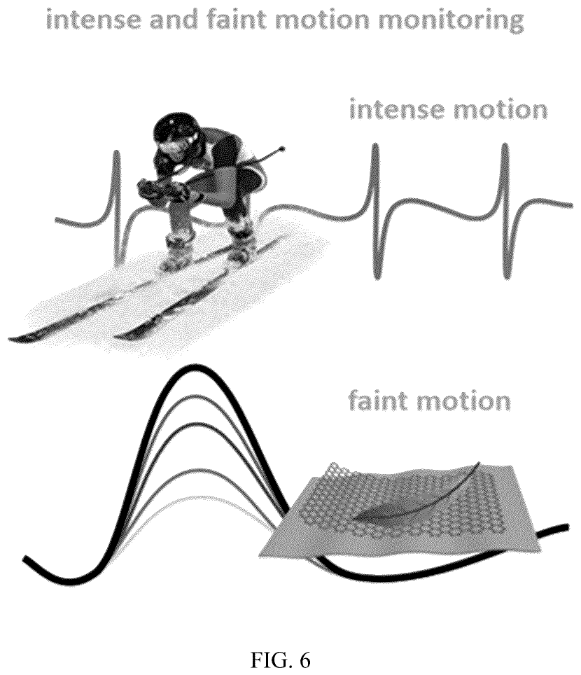

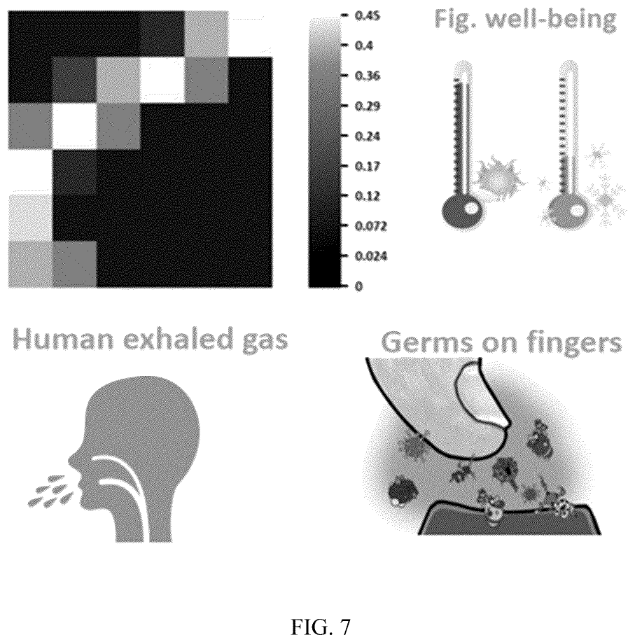

The crumpled graphene/NiS₂ nanocomposite patterns with 3D networks are created through facile direct laser writing and electrodeposition, followed by transfer printing onto a pre-strained elastomeric substrate. In brief, the interconnected porous laser-induced graphene (LIG) patterns prepared by irradiation of a thin polyimide (PI) film with a CO₂ laser (wavelength of 10.6 μm and laser power of 3.8 W) (FIGS. 1 and 3) can act as the working electrode for electrodeposition of the crystallized NiS₂ nanoparticles directly on LIG without any polymeric binder and conductive additive. Following transfer printing and the pre-strain strategy can then obtain crumpled porous LIG/NiS₂ nanocomposite films, with well controlled degree of crumple by the level of pre-strain (FIG. 5). The highly crumpled nanocomposite exhibits high stretchability and mechanical robustness, along with improved electrical conductivity owing to increased interfacial interactions between nanocomposites and charge carriers. Compared to other synthesis strategies, this method also allows uniform coating of active materials on the surface and throughout the depth of the porous LIG skeleton to load a higher mass of active materials and ensure enhanced mechanical adhesion and electrical contact even upon mechanical deformations. Additionally, this method allows facile control of the morphology, crystal structure, and physicochemical properties of the resulting LIG nanocomposites by simply adjusting the electrodeposition conditions, such as the ions in the solution, deposition rates, electrodeposition time, temperatures, pH values, and additives during the electrodeposition. For instance, adjusting the atomic percentage of Ni and S in the solution can yield Ni₃S₂, NiS, NiS₂, and NiₓS₆, with the particle size and distribution density controlled by the electrodeposition time. Therefore, the crumpled porous films can serve as a new class of stretchable electronic materials with adjustable surface structures and material properties for varying target applications, such as robotics, prosthetics, virtual reality and personalized healthcare (FIGS. 6 and 7).

### Structural and Composition Characterization of Crumpled Porous Graphene NiS₂ Nanocomposite

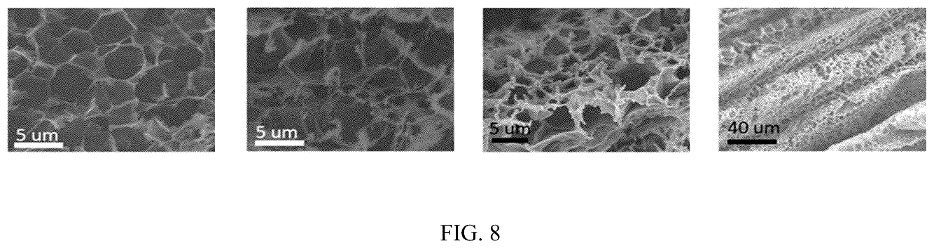

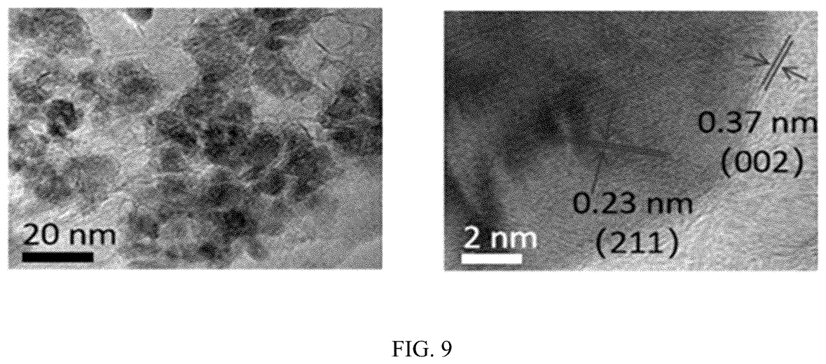

Characterization of the crumpled graphene/NiS₂ nanocomposite with scanning electron microscope (SEM), transmission electron microscopy (TEM), X-ray diffraction (XRD), X-ray photoelectron spectroscopy (XPS), and Raman spectroscopy reveals the structure and material composition. With few-layered graphene sheets interconnected to form a porous honeycomb cellular structure in the pristine LIG (FIG. 8), NiS₂ nanoparticles are uniformly grown on the surface of graphene sheets in the nanocomposite for enhanced accessible surface area and ion diffusion rate (FIG. 8). The typical surface morphology of the as-prepared samples after transfer printing (FIG. 8) and the full release of the pre-strain (FIG. 8) clearly reveals ripple-like crumped structures on the surface of the nanocomposite film with well-maintained 3D porous networks structure to afford high stretchability and specific surface area. Thin graphene flakes with ripple-like wrinkled structure observed in the TEM image (FIG. 9) are likely attributed to the thermal expansion during the pulsed CO₂ laser irradiation. NiS₂ nanoparticles with a relatively uniform size of ~20 nm also show porous structures for increased specific surface area and shorter transport pathways for both electrons and electrolyte ions. The lattice spacing of ca. 0.23 and 0.37 nm in the high-resolution TEM (HRTEM) image corresponds to the distance between two neighboring (211) planes in NiS₂ nanocrystals and (002) planes in graphitic materials, respectively (FIG. 9).

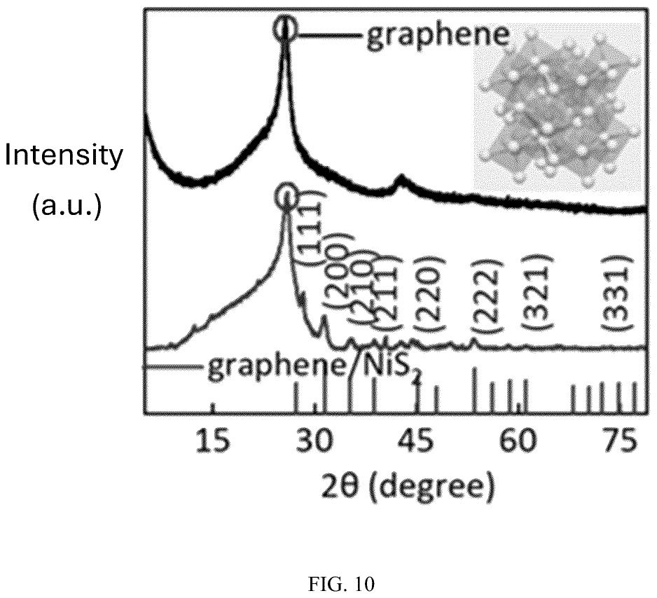

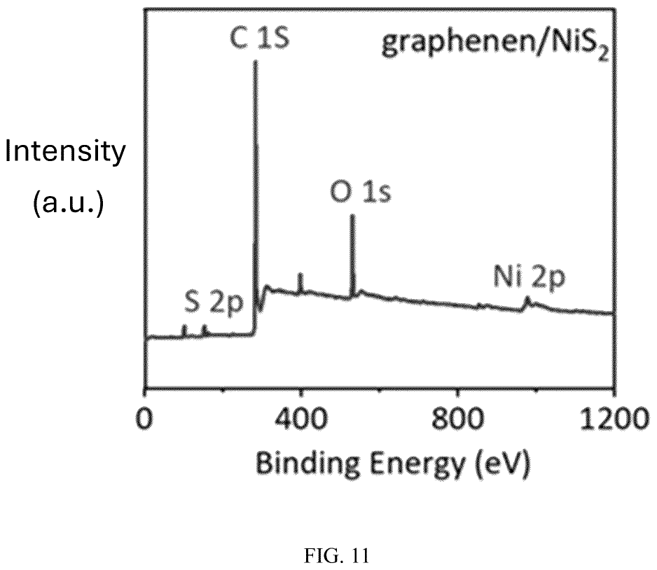

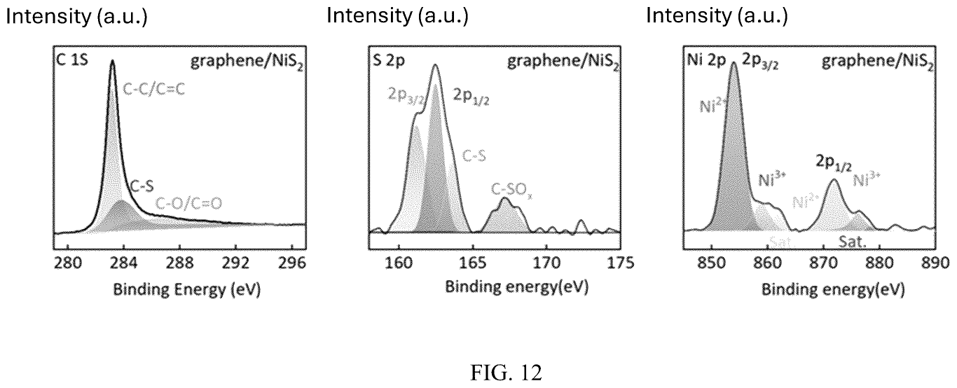

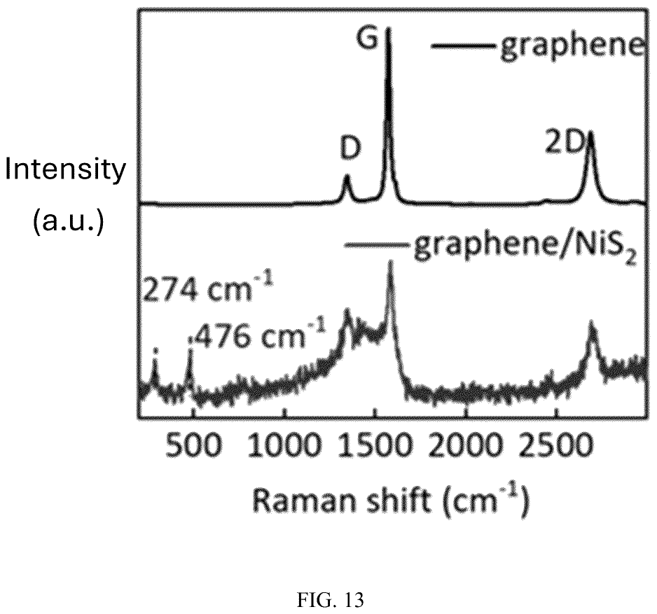

The broad diffraction peak located at 2θ=25.9 in the XRD pattern of the LIG foam (FIG. 10) corresponds to the graphite (002) plane, suggesting the high degree of graphitization in LIG. In addition to the typical peak originating from LIG, the porous graphene/NiS₂ nanocomposite also shows other well-defined diffraction peaks in the XRD pattern, which can be well indexed to the cubic phase of NiS₂ crystals (JCPDS Card No. 11-0099, space group Pa-3(205); a=b=c=5.67 Å; a=p=y=90°). The full survey XPS spectra of the porous graphene/NiS₂ nanocomposite (FIG. 11) mainly show carbon, sulfur, and nickel, along with a certain amount of oxygen resulting from the oxygen reaction on the surface of the nanocomposite. The C 1s XPS spectrum shows three major peaks with binding energies at 283.2, 283.9, and 286.9 eV (FIG. 12), corresponding to C—C, C—S, and C—O peaks, respectively. Moreover, two dominant peaks at 853.9 and 871.8 eV in the S 2p spectrum (FIG. 12) correspond to S 2p_{3/2} and S 2p_{1/2}, while two weak peaks at 161. eV and 163.9 eV correspond to a very small amount of NiS and organic sulfur, respectively. The high-resolution Ni 2p in the nanocomposite fits with Ni 2p_{1/2} (871.8 eV) and Ni 2p_{3/2} (853.9 eV) spin-orbit peaks and two shakeup satellite peaks at 878.9 and 862.5 eV (labeled as “Sat.”). Furthermore, three characteristic peaks at ~1351 (D bond), 2580 (G bond), and 2700 cm⁻¹ (2D bond) in the Raman spectrum of the LIG and porous graphene/NiS₂ nanocomposite (FIG. 13) further confirm the formation of crystalline graphene. The D/G intensity ratio of ~0.15 and 0.31 indicates a high degree of graphene structures in the as-prepared samples. Additionally, two prominent peaks at 274 and 476 cm⁻¹ correspond to NiS₂ in porous graphene/NiS₂ nanocomposite, indicating the successful growth of NiS₂ on the LIG scaffold.

### Physiochemical Properties of the Crumpled Porous Graphene NiS₂ Nanocomposite

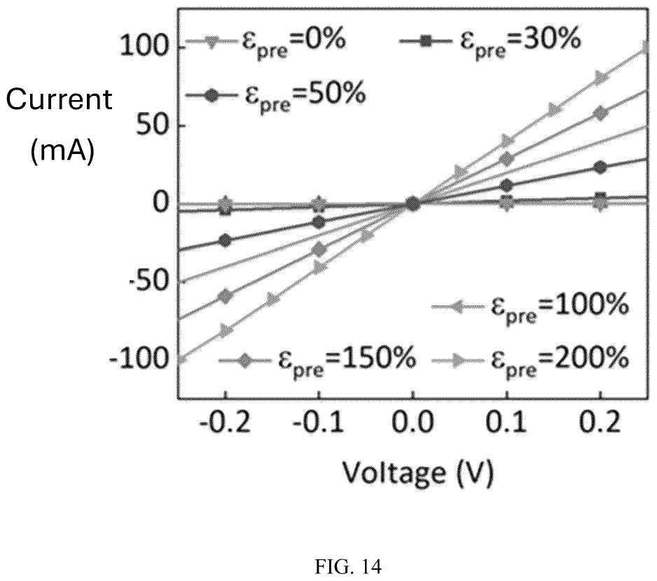

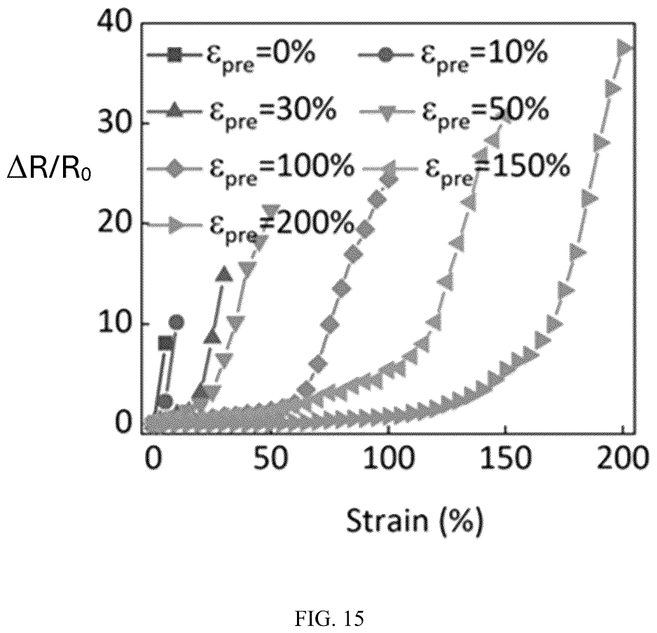

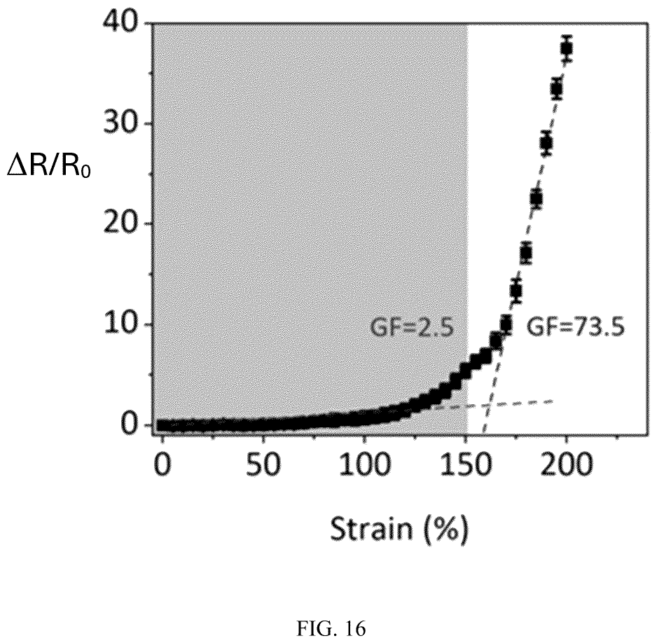

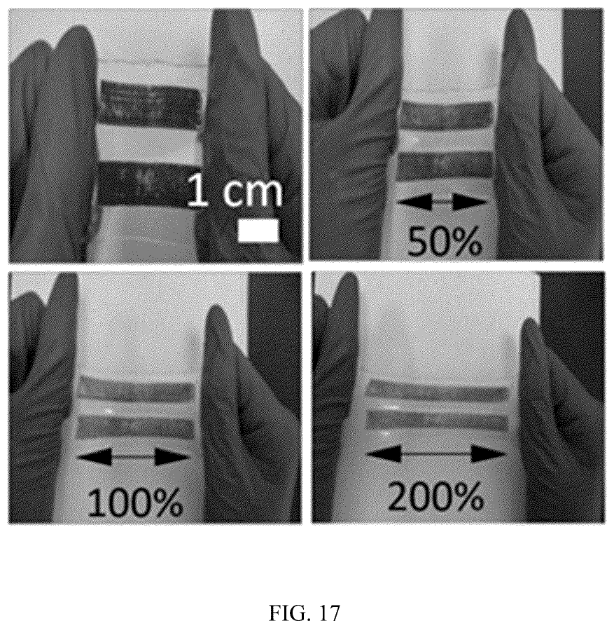

As the level of crumpled structure depends on the pre-strain εₚᵣₑ, the crumpled porous graphene/NiS₂ nanocomposite is prepared with εₚᵣₑ ranging from 0 to 200%. The linear current-voltage (I-V) curves of the crumpled porous graphene/NiS₂ nanocomposite (FIG. 14) indicate good ohmic contacts formed between NiS₂ nanoparticles and the LIG foam scaffold. Moreover, the increased slope of the I-V curve with the increasing εₚᵣₑ highlights the enhanced conductive pathways due to the increased effective contact area. Compared with the sheet resistance of 38 Ω/sq for the pristine LIG foam (εₚᵣₑ of 200%), porous graphene/NiS₂ nanocomposite and its crumpled form exhibit reduced values to 33 and 28 Ω/sq, suggesting enhanced electrical conductivity from the direct growth of metallic NiS₂ on the 3D network scaffold. The sheet resistance value of the crumpled porous graphene/NiS₂ nanocomposite with a pre-strain εₚᵣₑ of 200% is smaller than previously reported LIG, LIG foams with a variety of metals (Au nanocrystals), and metal oxides (Co₃O₄, MnO₂). Furthermore, the normalized resistance change (ΔR/R₀) of the crumpled porous graphene/NiS₂ nanocomposite from increased εₚᵣₑ exhibits enhanced stretchability and reduced changes with the increasing applied strain (FIG. 15). Meanwhile, the calibration curve of crumpled graphene/NiS₂ nanocomposite films with εₚᵣₑ of 200% is piecewise linear in two regions with extremely high gauge factor (GF, GF (ΔR/R₀)/ε) of 2.5 for strain 0-140%, and 73.5 for strain 140-200% (FIGS. 16 and 17). Although large GF is often preferred for practical application, overlarge GF under an applied strain ranging from 140% to 200% can lead to mass of small cracks in the crumpled graphene/NiS₂ nanocomposite films, which would lead to negatively affect the electronic conductivity and electromechanical performance. Therefore, the excellent working range of the crumpled graphene/NiS₂ nanocomposite films with εₚᵣₑ of 200% is 140%, and the nanocomposite films can fully recover to their original state under small strains.

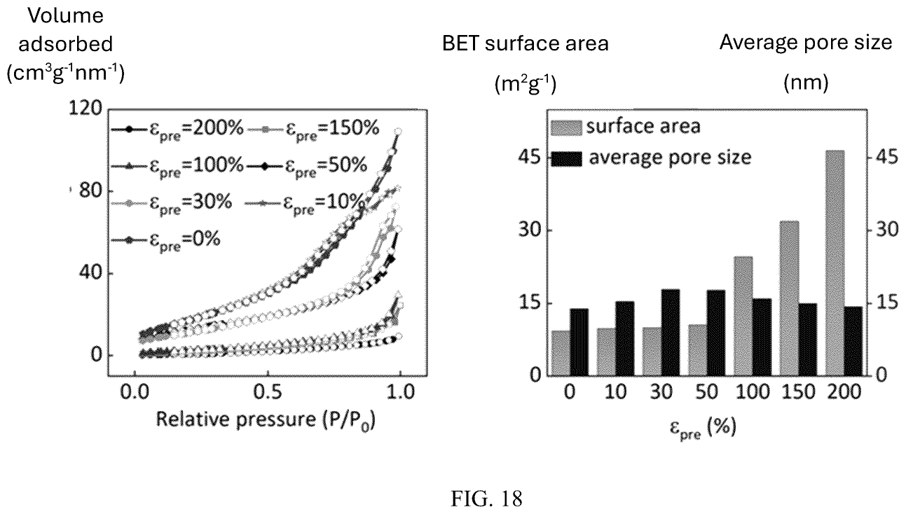

The pre-strain εₚᵣₑ can also be used to control the Brunauer-Emmett-Teller (BET) surface area and the pore size distribution. First of all, the hysteresis loop in the nitrogen adsorption/desorption isotherms of the porous graphene/NiS₂ nanocomposite with different pre-strain levels εₚᵣₑ ranging from 0 to 200% indicates porous structures in all samples (FIG. 18). As the pre-strain εₚᵣₑ increases from 0 to 200%, the surface area calculated from the Barrett-Joyner-Halenda (BJH) method gradually increases from 158 m² g⁻¹ to 217 m² g⁻¹, with the average pore size increased first and then decreased. The increased surface area with εₚᵣₑ can be attributed to the increased wavy structure formed in the crumped porous graphene/NiS₂ nanocomposite. Furthermore, the water contact angle decreases from 40° for the pristine LIG foam to 310 for the crumped porous graphene/NiS₂ nanocomposite with εₚᵣₑ of 200%, suggesting improved surface wettability due to the electrodeposited NiS₂ nanoparticles for faster adsorption of ionic liquids and enhanced electrochemical energy storage.

### Output Performance of the Crumpled Porous Graphene NiS₂-Based Stretchable TENG

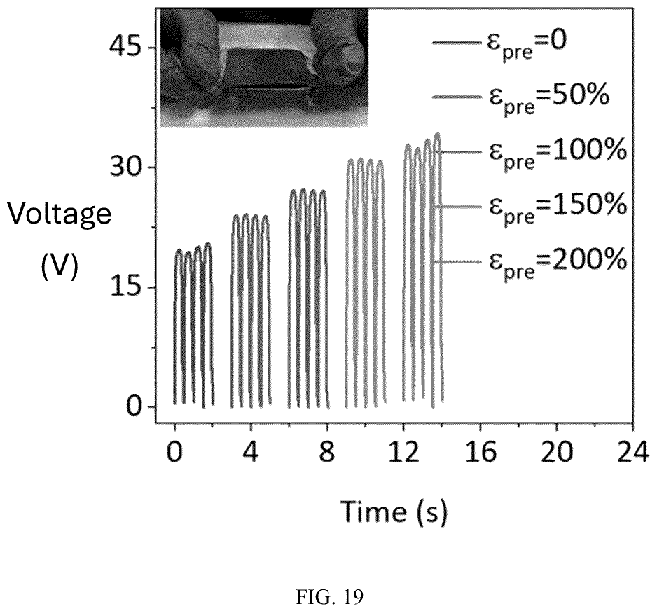

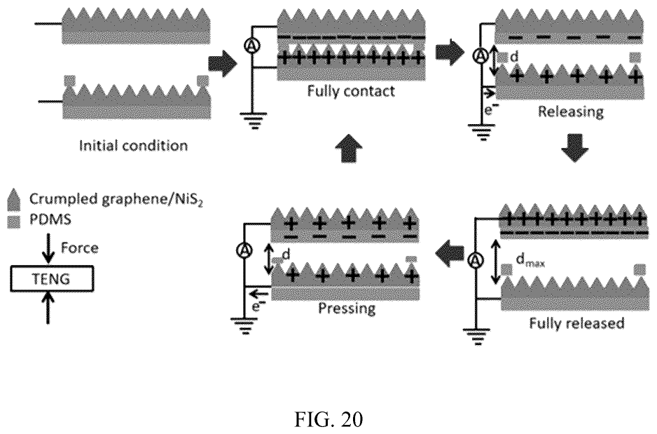

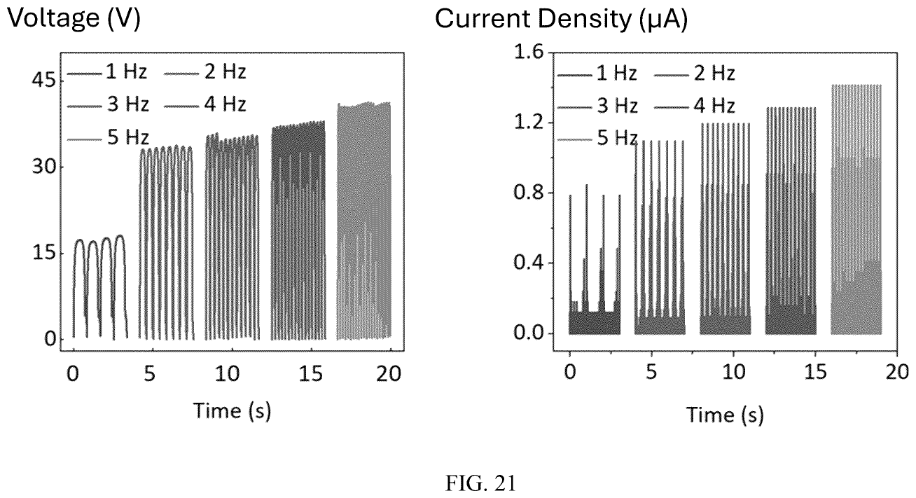

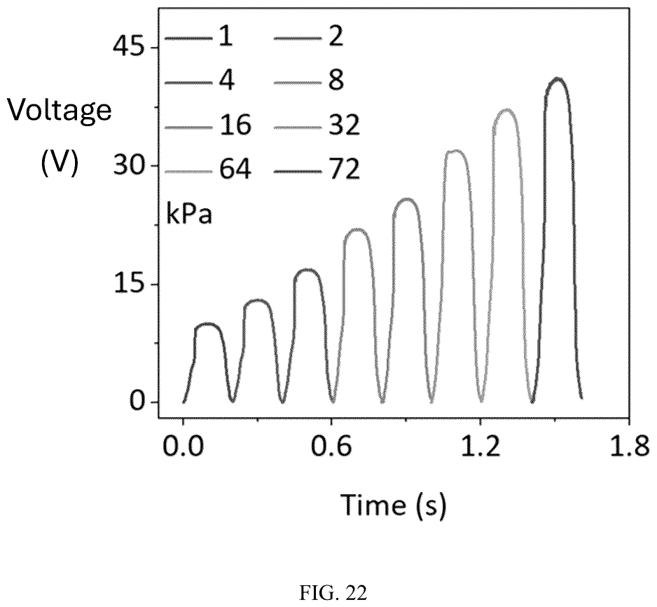

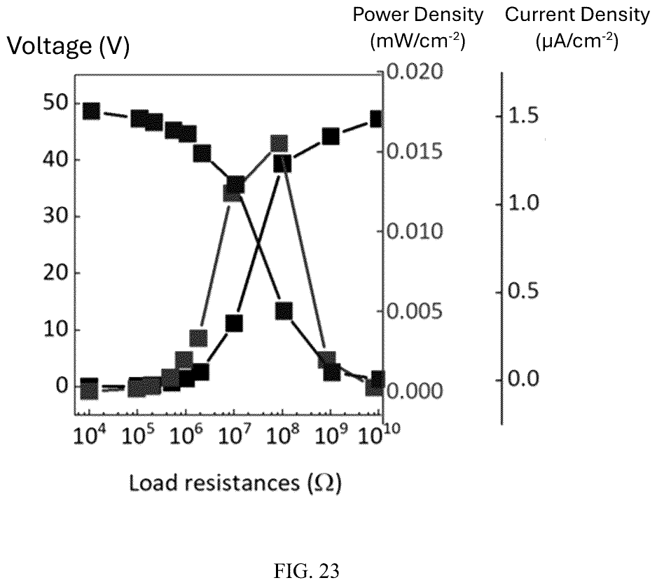

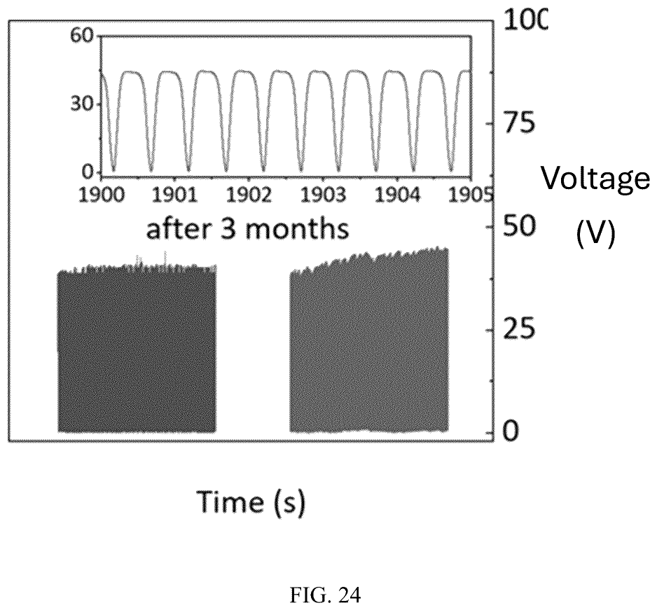

The crumped porous graphene/NiS₂ nanocomposite with highly conductive and stretchable properties as a top electrode can be combined with a bottom triboelectric polydimethylsiloxane (PDMS) layer (with four PDMS cylindrical spacers) to yield an intrinsically stretchable TENG (FIG. 19). As the TENG utilizes contact electrification coupled with electrostatic induction to convert mechanical energy into electricity (FIG. 20), triboelectric charges generated from cyclic mechanical motion flow through external circuits to maintain electrostatic equilibrium, generating an alternating current output. The efficiency of the TENG depends on the difference in the electron affinities of the two electrode materials and the microstructures of the contact surfaces, so the level of the pre-strain εₚᵣₑ plays a critical role in the performance of the stretchable TENG. As the pre-strain εₚᵣₑ increases from 0 to 200%, the output voltage of stretchable TENG (at a frequency of 2 Hz) monotonically increases from 19 to 34 V, which is attributed to increased surface roughness and effective contact area (FIG. 19). Therefore, the pre-strain εₚᵣₑ of 200% is chosen in the following studies unless specified otherwise. As the deformation frequency increases from 1 to 5 Hz (at a pressure of 72 kPa), the output current (and voltage) increases from 0.8 to 1.4 μA (and from 18 to 42 V) (FIG. 21), resulting from a higher flow rate of charges. In addition, the output voltage also increases from 10 to 41 V as the pressure increases from 1 to 72 kPa (FIG. 22), which is attributed to the increased contact area between the two triboelectric electrodes. By using a load resistor from 10 kΩ to 10 GΩ, the output voltage (or current density) of the TENG sharply increase (or decreases) to result in a peak output power density of 1.6×10⁻² mW cm⁻² at a resistance of 2 MΩ (FIG. 23). The crumped porous graphene/NiS₂-based stretchable TENG also demonstrates excellent mechanical stability with negligibly small changes in the output voltage over three months (FIG. 24).

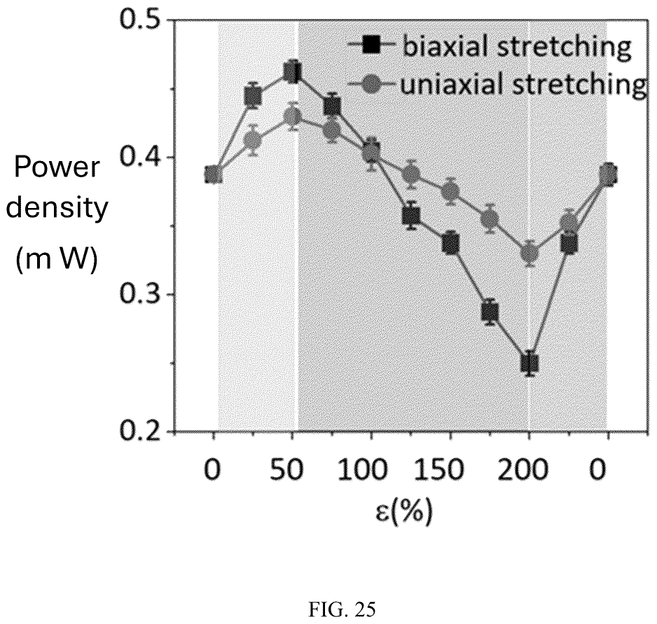

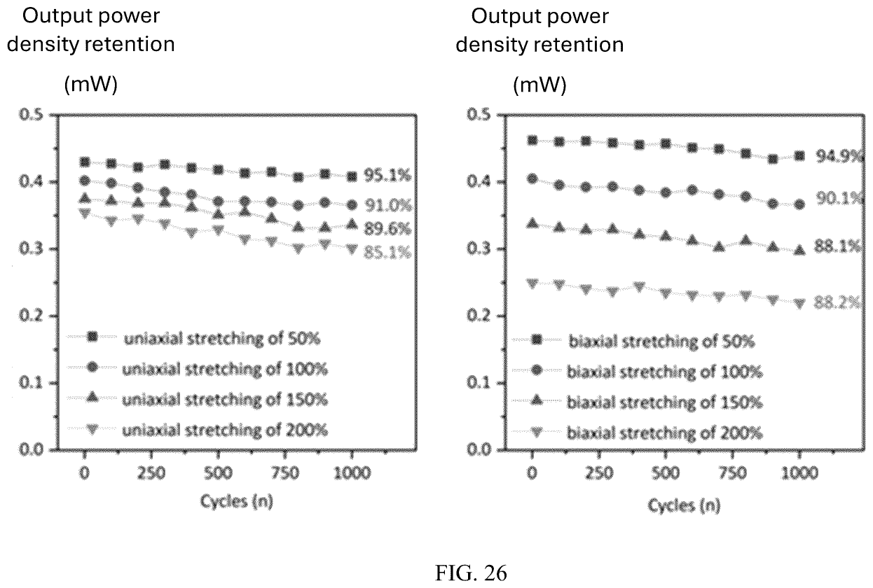

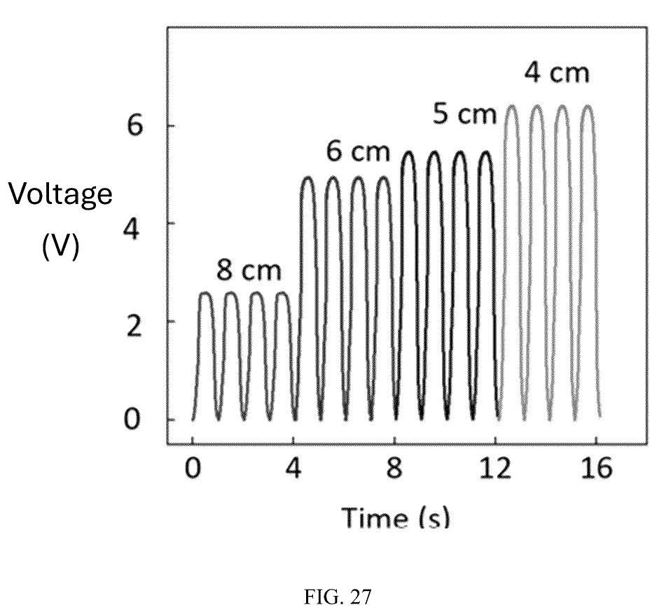

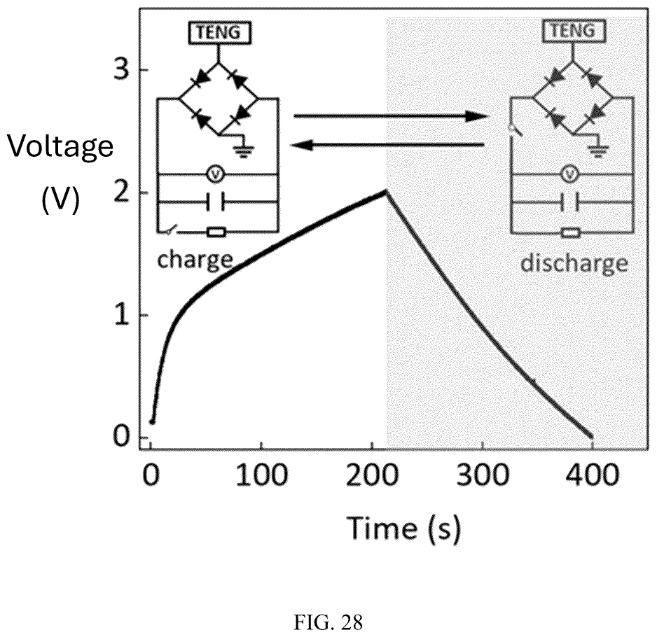

As the biaxial (or uniaxial) strain increases from 0 to 50% and then to 200%, the maximum output power of the device first increases from 3.9×10⁻¹ to 4.6×10⁻¹ (or 4.3×10⁻¹) mW but then reduces to a minimum value of 2.5×10⁻¹ (or 3.3×10⁻¹) mW with 64% (or 85%) output power retention (FIG. 25). The initial increase results from increased effective contact area between the crumpled graphene/NiS₂ and PDMS layer upon stretching, while the subsequent decrease is likely attributed to the increased resistance of the crumpled graphene/NiS₂ electrode. The highly elastic property of the TENG also allows the recovery of the structure and output power density to the initial state after the tensile strain is fully released. The stretchable TENG also shows a stable maximum output power density over 1000 cycles under various biaxial and uniaxial strains (FIG. 26). As the bending at a radius of 8/6/5/4 cm, the output voltage (or peak output power density) increases from 2.7 to 6.1 V (or from 0.67×10⁴ to 3.4×10⁻⁴ mW cm⁻²) (FIG. 27), resulting from the higher input mechanical energy and the effective contact area. By using a bridge rectifier, the intrinsically stretchable TENG (at 72 kPa) can charge a commercial capacitor (NCC200, 2V, 100 ηF) (FIG. 28) and low-power consumer electronics. The excellent output performance of the crumpled porous graphene/NiS₂-based stretchable TENG compares favorably with the previously reported TENGs (Table 1).

TABLE 1

| | peak output power | | Elongation |
|---|---|---|---|
| Electrode materials | density | | (%) |
| crumpled porous | 1.6 × 10⁻² | mW cm⁻² | 200 |
| graphene/NiS₂ // PDMS | | | |
| PAAm-LiCl hydrogel // PDMS | 0.35 × 10⁻² | mW cm⁻² | 1160 |
| or VHB | | | |
| 3-ply-twisted stainless | 0.85 × 10⁻² | mW cm⁻² | 60 |
| steel/polyester fiber blended | | | |
| yarn | | | |
| PDMS-silicone/ionic solution | 0.18 × 10⁻² | mW cm⁻² | 60 |
| PDMS // crumped Au | 0.22 | mW cm⁻² | 100 |
| PDMS // crumped graphene | 0.25 | mW cm⁻² | 120 |

Electrochemical Performance of the Crumpled Porous Graphene/NiS₂-based MSCAs

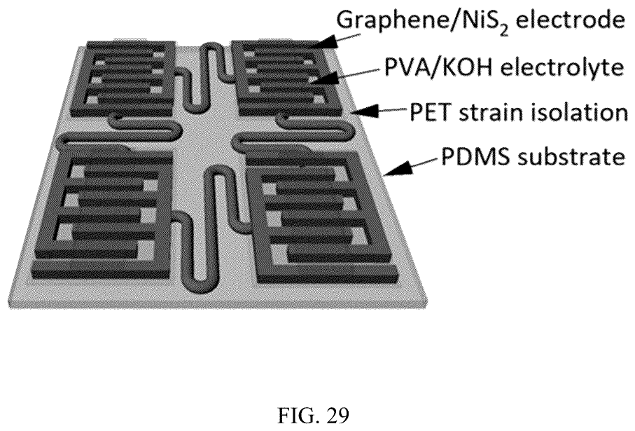

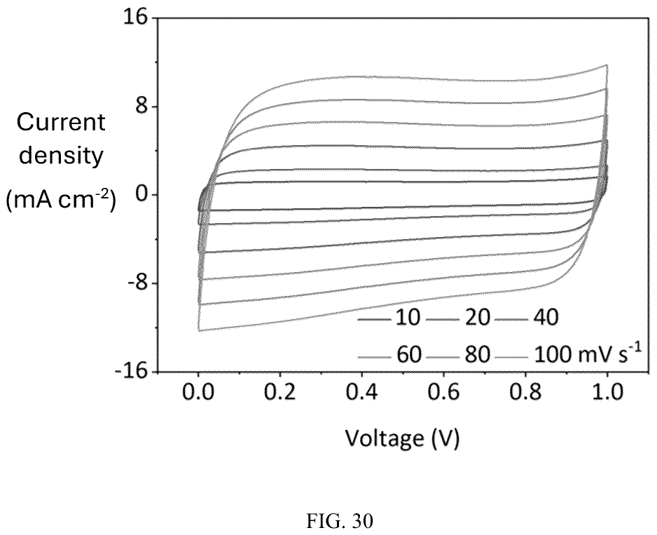

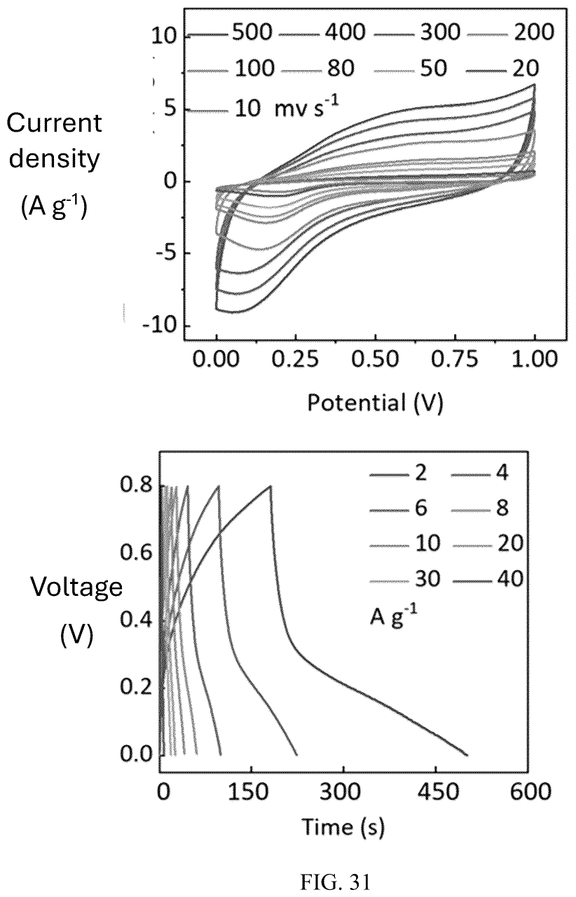

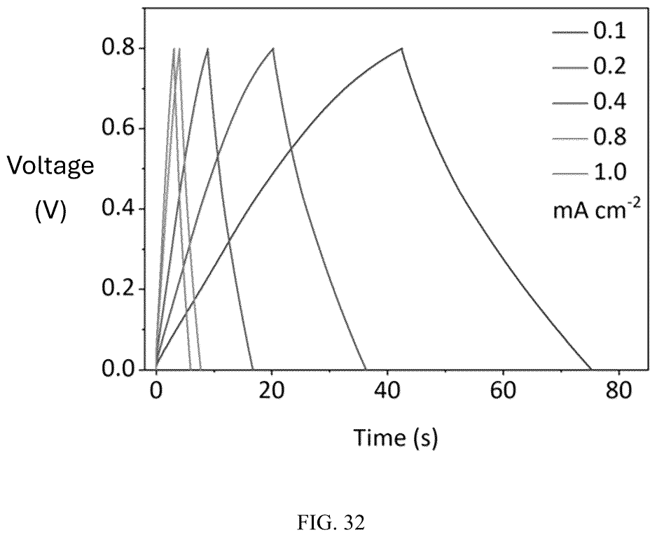

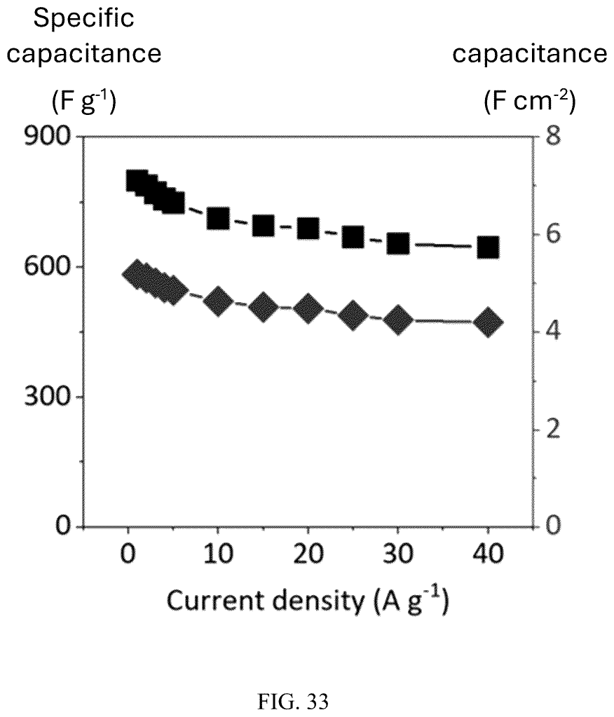

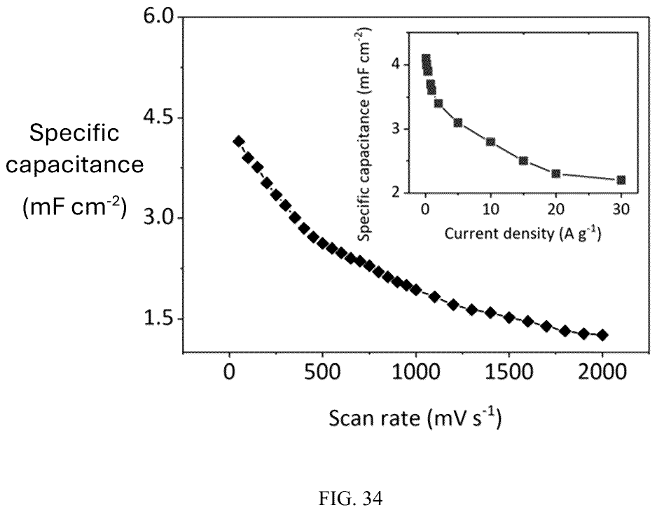

With superior structural and electronic properties, the crumpled porous graphene/NiS₂ nanocomposite can act as interdigitated electrodes, current collectors, and serpentine interconnections in an all-in-one planar micro-supercapacitor array (MSCA) with polymeric gel electrolyte (PVA/KOH) and island-bridge layout (FIG. 29). Compared with pristine LIG-based MSC cell with nearly rectangular shape as double-layer capacitor (FIG. 30), crumpled porous graphene/NiS₂-based MSC shows a distinct pair of redox peaks during the anodic and cathodic sweeps in cyclic voltammetry (CV) (FIG. 31). The redox peaks result from the faradic oxidation of NiS₂ with the alkaline electrolyte: NiS₂+3OH⁻↔NiS₂(OH)₃+3e⁻. As the scan rate increases, the current density increases and the reduction/oxidation peaks move to more positive/negative potential (FIG. 31), indicating the relatively low internal resistance and good high-rate capability of the electrode. In addition, the galvanostatic charge-discharge (GCD) curves of the porous graphene/NiS₂-based MSC at different current densities show significantly increased discharging time compared to that of pristine LIG-based MSC cell (FIG. 32), further suggesting larger charge capacity from NiS₂ nanoparticles (FIG. 31). Furthermore, nonlinear charge/discharge curves further corroborates rapid Faradaic pseudocapacitance reactions to govern charge storage. Despite strong pseudocapacitive behavior suggested by the GCD curves, the charge/discharge curves are approximately symmetric, indicating a superior reversible redox reaction and good Coulombic efficiency. The specific areal (or gravimetric) capacitance Cₐ (or C_{g}) of the porous graphene/NiS₂-based MSC only decreases from 5.19 F cm⁻² (or 799 F g⁻¹) to 4.20 F cm⁻² (or 646 F g⁻¹) (with rate capability of 80.1%) as the current density increases from 1 to 40 A g⁻¹ (FIG. 33). Collectively, the porous LIG/NiS₂-based MSC with high specific areal (or gravimetric) capacitance and maximum energy density compares favorably over the other LIG-based MSCs (FIG. 34, Table 2).

TABLE 2

| Electrode | Specific areal | Rate capability (the | Maximum energy |
|---|---|---|---|
| materials | (or gravimetric) | capacitance retention) | density |
| crumpled porous | 5.19 F cm⁻² (799 | 80.1% (the current | 49.89 Wh kg⁻¹ at a |
| graphene/NiS₂ | F g⁻¹) at 1 A g⁻¹ | increased from 1 to 40 A | power density of |
| | | g⁻¹) | 0.25 kW kg⁻¹ |
| Co₃O₄@LIG | 3.0 mF cm⁻² | 71.5% (the current | 19.9 Wh kg⁻¹ at a |
| | (143 F g⁻¹ at 1 | increased from 1 to 40 A | power density of |
| | A g⁻¹) | g⁻¹) | 0.5 kW kg⁻¹ |
| TiO₂-graphene | 6.8 mF cm⁻² at 5 | 47.1% (the current | 0.22 μWh/cm² and |
| | μA | increased from 5 μA to 20 | 39 μW/cm² |
| | | μA) | |
| Laser-scribed | 3.05 mF cm⁻² | 60% (the current | 2.1 mWh cm⁻³ at |
| graphene (LSG) | at 16.8 mA cm⁻³ | increased from 16.8 mA | 0.01 V s⁻¹; 1.37 |
| | | cm⁻³ to 1.84 × 10⁴ mA | mWh cm⁻³ at 1 V s⁻¹ |
| | | cm⁻³) | |
| graphene | 80.7 μF at 200 | 97.8% (the scan rate | 0.14 mWh cm⁻³ at a |
| | mV s⁻¹ | increased from 200 mV s⁻¹ | power density of |
| | | to 1000 mV s⁻¹) | 495 W cm⁻³ |
| PANI/nitrogen- | 261.85 mF cm⁻² | 97.55% (the current | 11.40 μWh cm⁻² at |
| doped graphene | at 0.5 mA cm⁻² | increased from 0.5 mA | 640 μWcm⁻² |
| quantum dots | | cm⁻² to 1 mA cm⁻²) | |

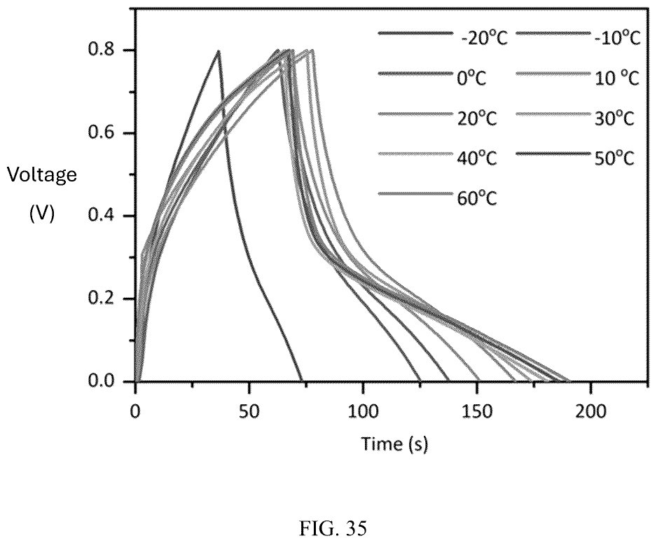

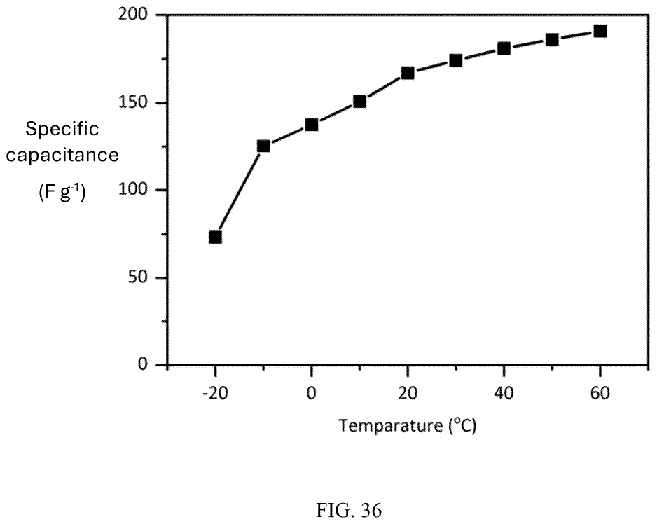

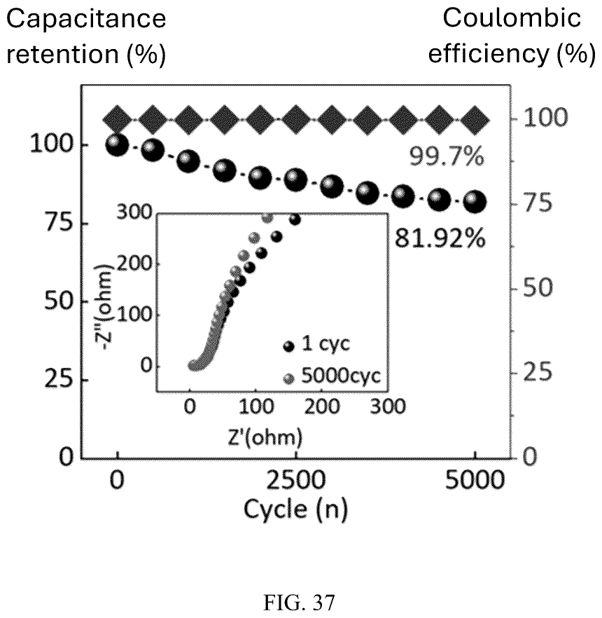

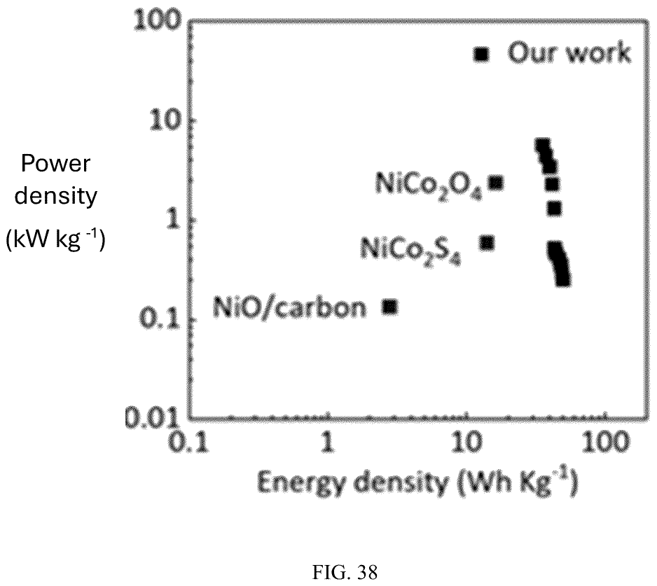

The GCD charging and discharging curves of the crumpled porous graphene/NiS₂-based MSC in the temperature range from −20 to 60° C. are approximately symmetric, indicating a good electrochemical capacitive characteristic and excellent reversible redox reaction of the porous graphene/NiS₂ electrodes (FIG. 35). Meanwhile, the slight curvature in the discharge curve suggests a balanced contribution from both pseudocapacitance and the double layer capacitance. As the temperature increases from −20° C. to 60° C., the charge storage capacity of the device is enhanced by about 160% (FIG. 36), which may be attributed to fast reaction kinetics of the crumpled porous graphene/NiS₂ electrodes and improved mobility of the electrolyte ions. These results demonstrate that the prepared MSCAs based on the crumpled porous graphene/NiS₂ can be used in a wide temperature range. The composite-based MSC can also retain ~81.92% of its initial capacitance even after 5,000 CV cycles, with superior Coulombic efficiency of 99.7% (at a constant current density of 3 A g⁻¹) to indicate excellent cycling stability (FIG. 37). The comparison in the electrochemical impedance spectroscopy (EIS) of the device before and after 5,000 charge-discharge cycles only shows a slightly increased faradaic charge-transfer resistance (Rₜ) and the equivalent series resistance (Rₛ). The good stability of the device over cycling results from the high electrical conductivity and excellent ion diffusion within the porous crumpled porous graphene/NiS₂ electrode. Considering the total device mass (i.e., electrodes, current collectors, and electrolyte), the nanocomposite-based MSC delivers a maximum energy density of 49.89 Wh kg⁻¹ at a power density of 0.25 kW kg⁻¹, with the highest power density of 5.70 kW kg⁻¹ at the energy density of 35.61 Wh kg⁻¹. The power and energy densities of the device are superior to the previously reported MSCs in the literature, including NiCo₂S₄ (31.5 Wh kg⁻¹ at 0.156 kW kg⁻¹), NiCo₂O₄ (34.9 Wh kg⁻¹ at 0.875 kW kg⁻¹), and NiO/carbon (10.2 Wh kg⁻¹ at 0.025 kW kg⁻¹) as revealed in the Ragone plot (FIG. 38).

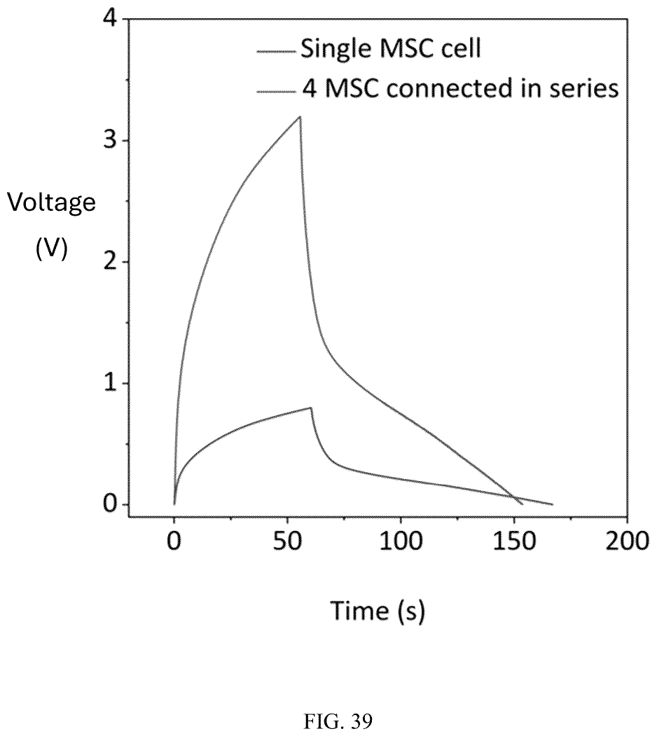

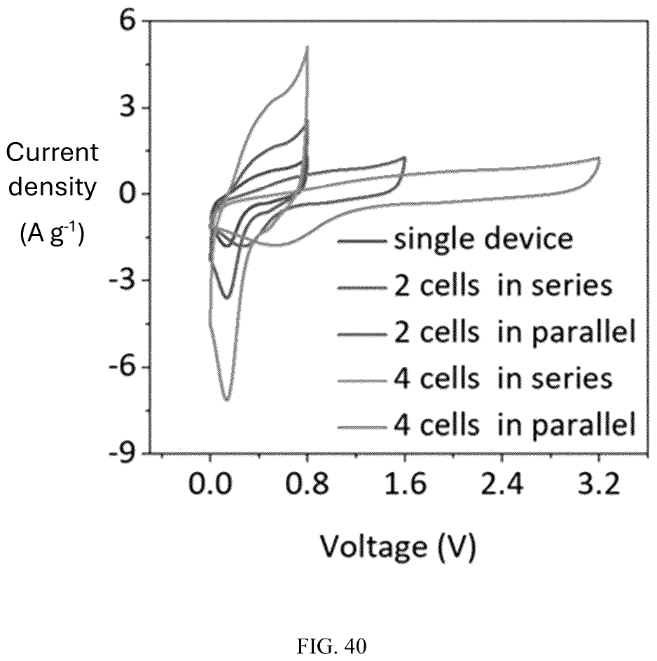

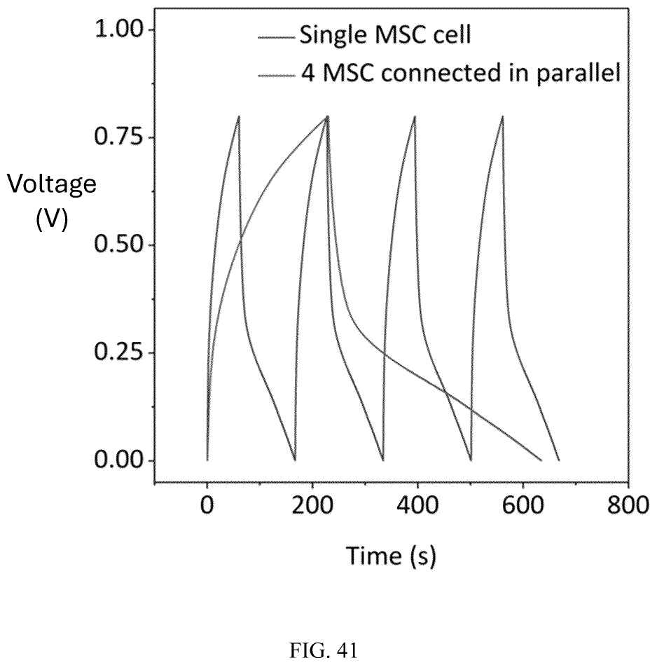

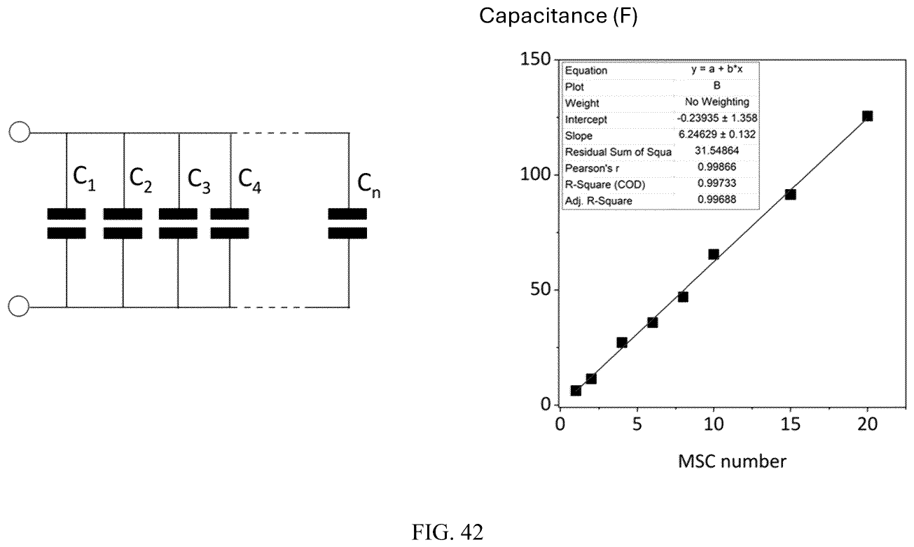

Connecting MSC cells in series and/or in parallel in the all-in-one planar MSCA can provide tunable output voltage and current. Compared with a single MSC cell with an output voltage window of 0.8 V, the MSCAs with two or four MSC cells connected in series can deliver an output working voltage window of 1.6 or 3.2 V with similar charge/discharge time (FIGS. 39 and 40). Similarly, the output current of the MSCAs with two or four MSC cells connected in parallel is increased by a factor of two or four, with the charge/discharge time increased by two or four times as well (FIGS. 40 and 41). Moreover, the overall capacitance of the MSCA is linearly proportional to the number of MSC cells in the array (FIG. 42), demonstrating excellent structural uniformity and scalability.

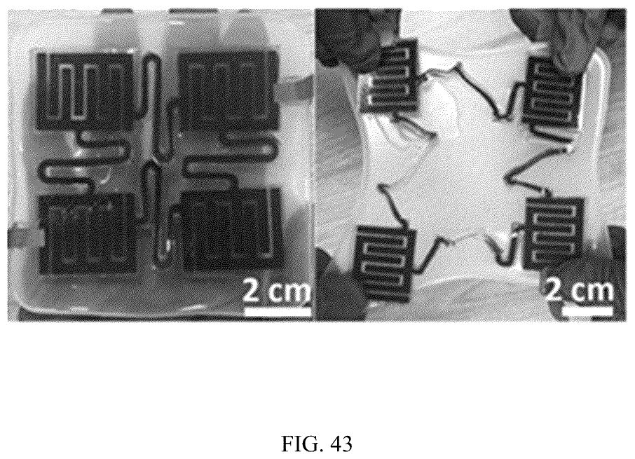

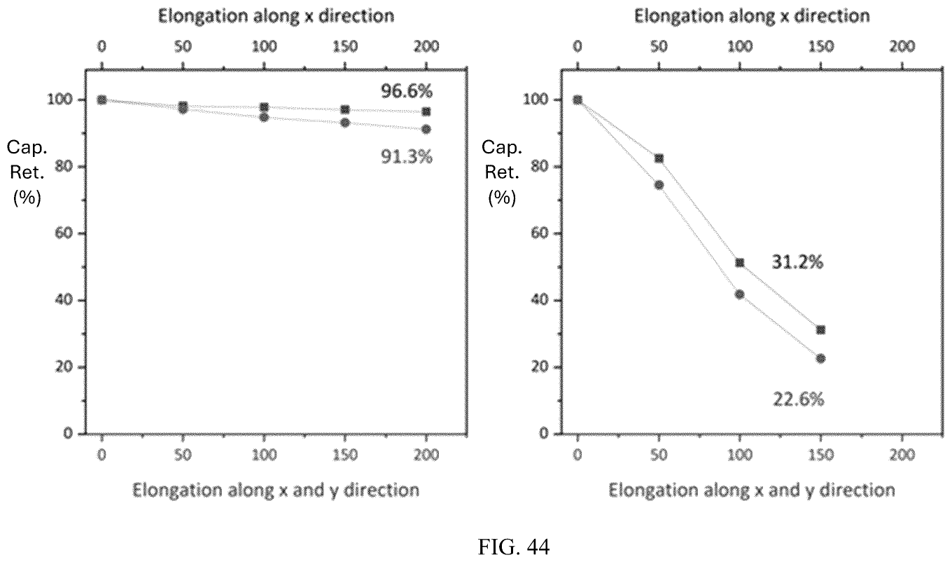

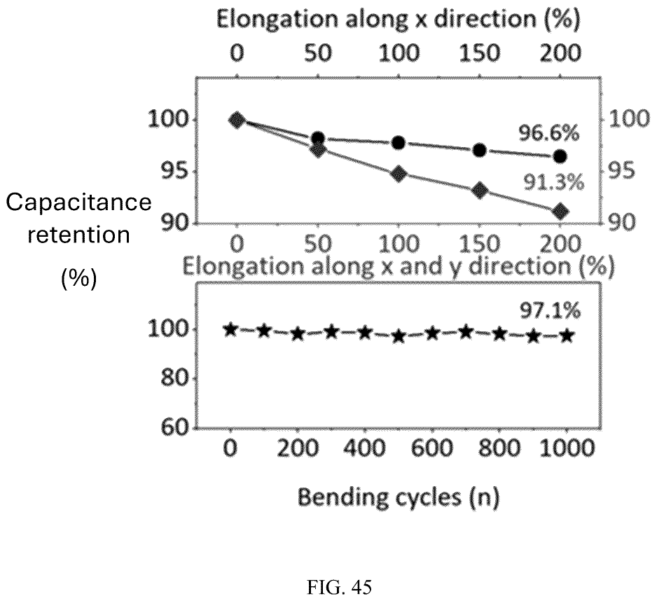

The islands-bridge layout together with the strain isolation design provided by a relatively rigid polyethylene terephthalate (PET) film between the MSC cell and stretchable PDMS substrate allows the MSCA to maintain stable electrochemical performance upon stretching (FIG. 43). The strain isolation and island-bridge design allow the MSCA to be stretched 50% biaxially (FIG. 43). The combination of the intrinsically stretchable crumpled graphene/NiS₂ nanocomposites with serpentine interconnects further increases the stretchability (FIG. 44). The stretchable MSCA with four MSC cells connected in parallel shows a small capacitance change of 3.4% (or 8.7%) even for uniaxial (or biaxial) stretching of 200% (FIG. 45) and it also retains more than 97.1% of its initial capacitance after bending of 180° over 1,000 cycles. These results clearly demonstrate that the as-prepared stretchable MSCAs exhibit high mechanical and electrochemical stability, which are highly promising for flexible electronics.

Sensing Performance of the Crumpled Porous Graphene NiS₂-based Wearable Sensors

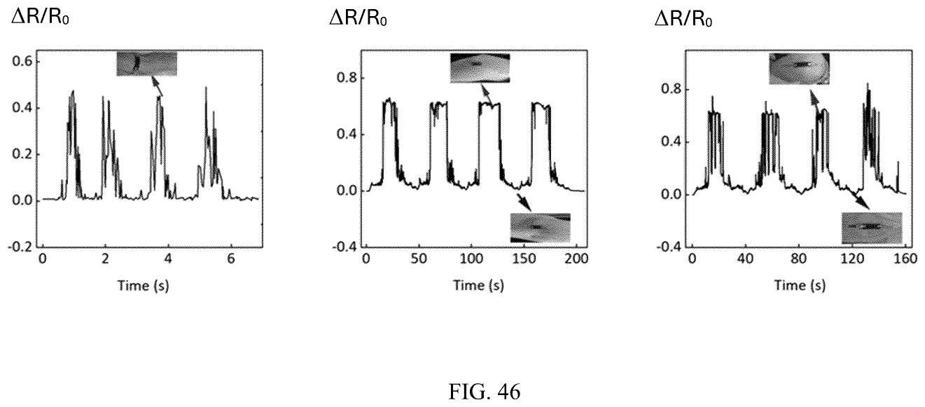

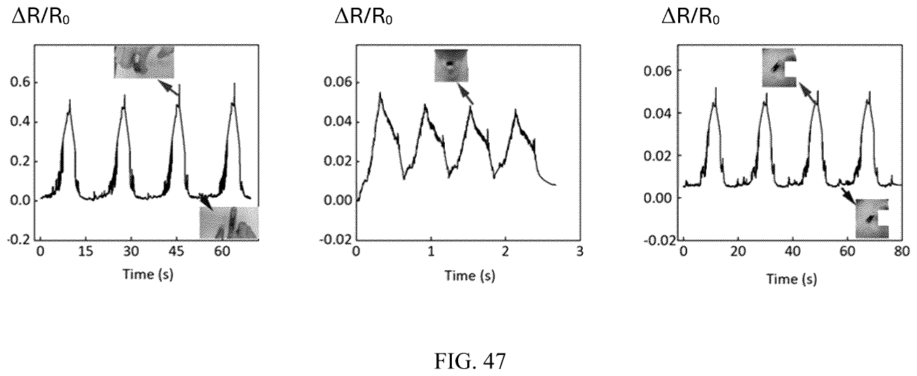

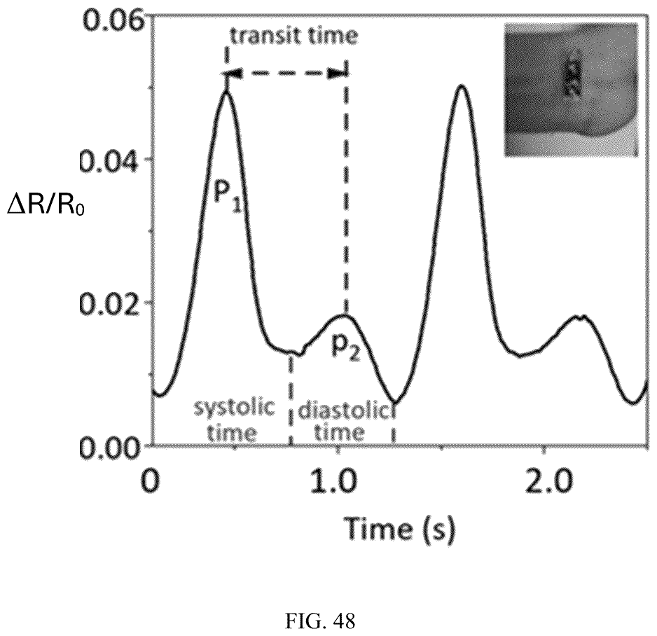

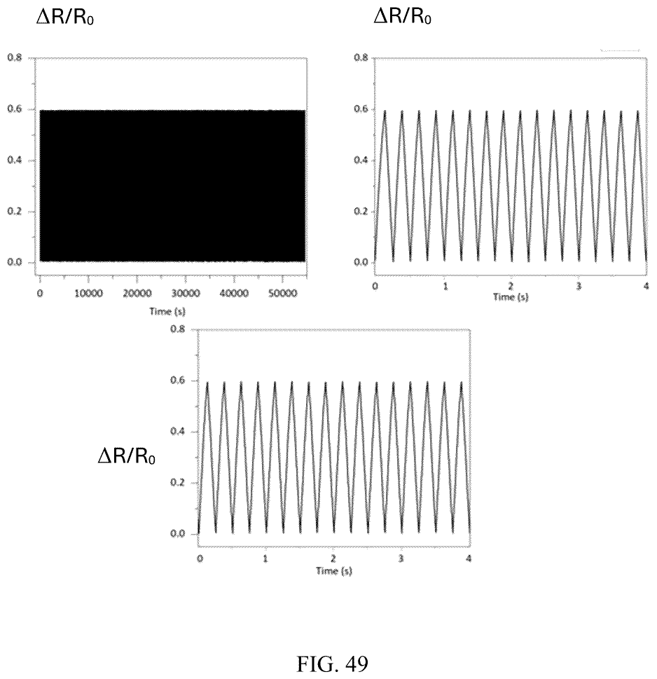

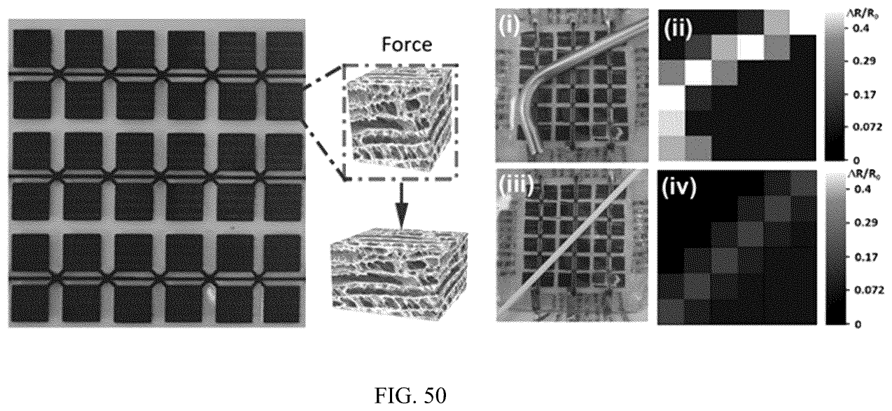

As the crumpled porous graphene/NiS₂ nanocomposite in 3D network is sensitive to the deformation caused by external pressure/strain, its seamless and conformal contact with human skin, with the help of a biocompatible liquid bandage (Nexcare, 3M), allows for accurate and long-term measurements of body motion. The strain sensor based on a single sensing unit attached to the joint (FIG. 46) or muscle (FIGS. 46 and 47) monitors the subtle motion from human activities. The porous 3D networked microstructure upon strain such as bending or stretching results in varied resistance of the crumpled porous graphene/NiS₂ nanocomposite. Attaching the strain sensor (3.0 mm×20.0 mm²) to the ankle noninvasively detects the human joint motion (FIG. 46) to monitor the walking frequency. Similarly, the sensor attached to the elbow (FIG. 46), knee (FIG. 46), and fingers (FIG. 47) can detect the bending-release movement of the elbow, knee, and fingers. Besides the large motions, the wearable sensor can also detect subtle human activities, including swallowing (FIG. 47), eye blinking (FIG. 47), and arterial pulse (FIG. 48). The measured radial artery pulse exhibits distinguishable systolic peak (“P₁”) and diastolic peak (labeled as “P₂”), with an average value of 72 beats per minute that is consistent with the measurement from a commercial sphygmomanometer (Omron, J760). In addition to excellent mechanical durability and stability over 55,000 loading-unloading cycles (1 kPa at 4 Hz) (FIG. 49), the sensor fabricated from the low-cost and scalable method can be facilely configured into an array of 6×6 to detect pressure distribution from a bent metal bar and a straight beverage straw. By employing the piezoresistive mechanism (FIG. 50), the applied external pressure increases the conductive pathways and reduces the total resistance. As a result, the as-prepared 6×6 sensor array can detect the shape of the metal bar (and beverage straw) with the color contrast mapping to illustrate the distribution of pressure (FIG. 50).

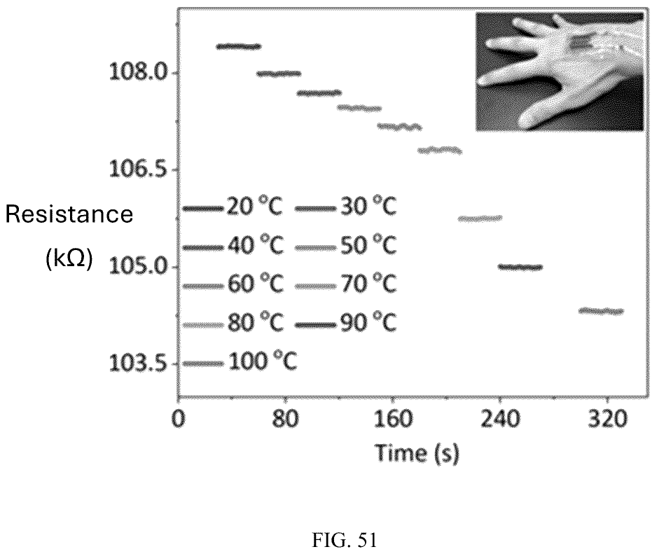

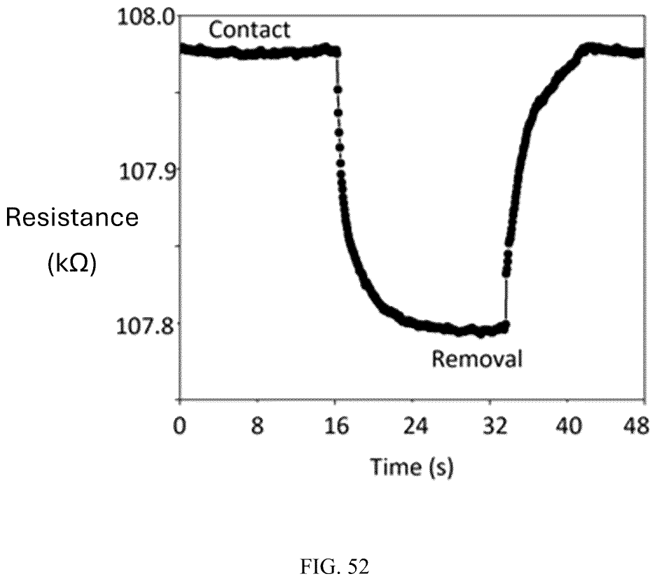

As the electron-phonon scattering and thermal velocity of electrons in the nanocomposite increase with the rising temperature, the increased charge conductivity of the crumpled porous graphene/NiS₂ in the serpentine shape can detect the corresponding temperature rise. With the temperature sensor calibrated by a commercial thermometer (Deli, LE505) in the range from 20 to 100° C. (FIG. 51), the approximately linear relationship between the relative resistance change and the temperature leads to a sensitivity value of S=3.38×10⁻⁶° C.⁻¹ (R²=0.999). Attaching/removing the wearable temperature sensor on/from the human wrist measures a response/recovery time of 10.5/8.5 s (FIG. 52), which compares favorably with that of 15.5/12.5 s and 10.5/9.5 s from other wearable temperature sensors based on LIG and indium oxide.

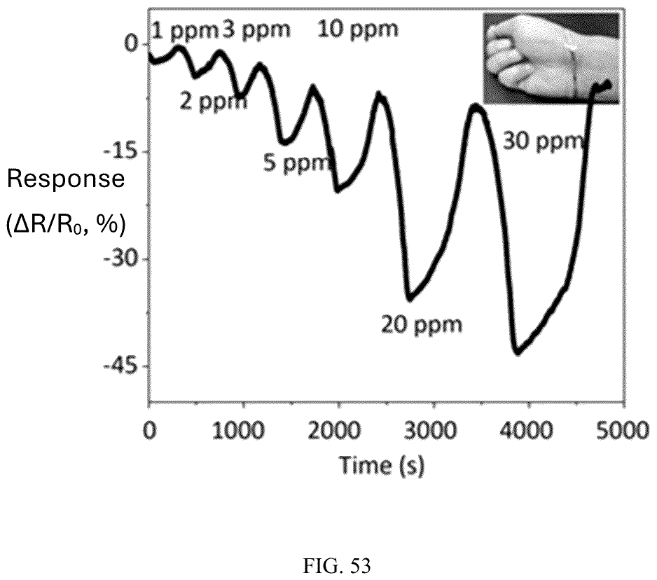

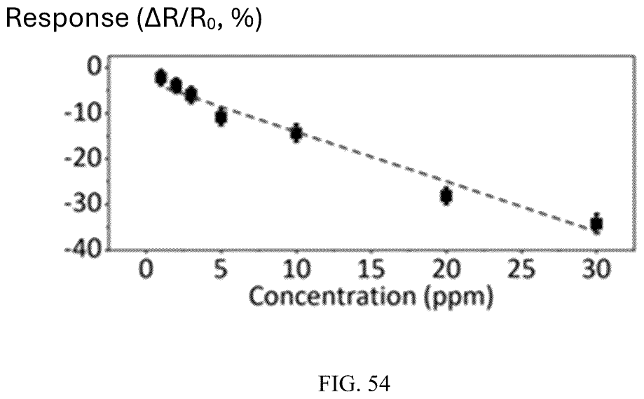

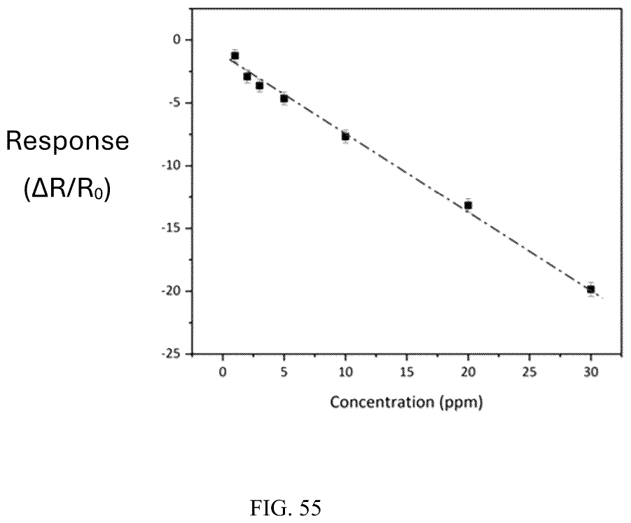

The crumpled porous graphene/NiS₂ also allows it to detect NO₂ with varying concentrations from 1 to 30 ppm at room temperature (FIG. 53). Meanwhile, the gas sensor exhibits a fast response/recovery time of 160/187 s to 1 ppm NO₂ even at room temperature. The sensitivity of 1.12 ppm-1 (R²=0.969) obtained from the linear fit between the response and NO₂ concentration (FIG. 54) is higher than that of 0.65 ppm⁻¹ (R²=0.953) from LIG-based gas sensor (FIG. 55). The crumpled porous LIG/NiS₂-based gas sensors with high sensitivity and rapid response/recovery also compares favorably over many previously reported NO₂ gas sensors (Table 3).

TABLE 3

| | Sensitivity | Recover time | Recover time |
|---|---|---|---|
| Functional materials | (ppm⁻¹) | (s) | (s) |
| crumpled porous | 1.12 | 160 | 187 |
| graphene/NiS₂ | | | |
| Sulfonated rGO | 0.443 | 675 | 850 |
| ethylenediamine- | 0.159 | 600 | 800 |
| modified rGO | | | |
| MoS₂ | 0.32 | 170 | 1630 |
| MoS₂@rGO | 0.06 | 360 | 720 |
| rGO/Co₃O₄ | 0.03 | 300 | 2400 |
| rGO | 0.05 | 240 | 240 |

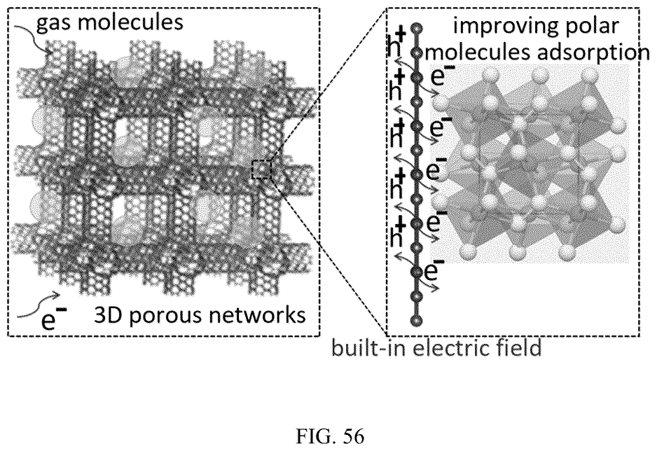

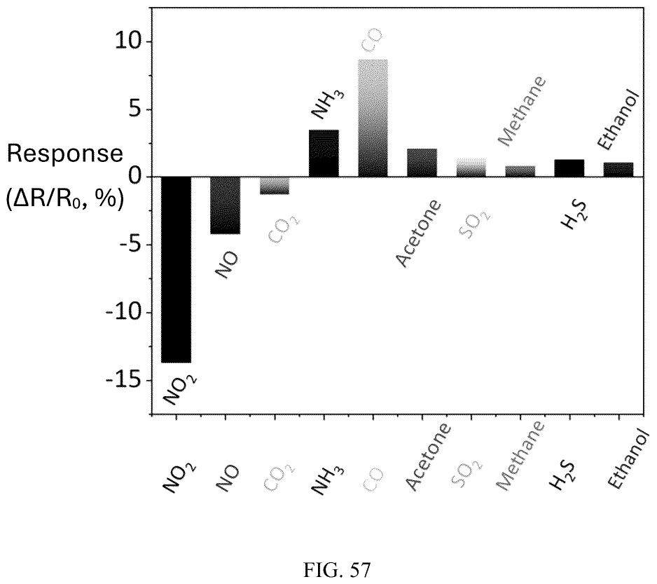

The high sensitivity of the gas sensor may be attributed the unique 3D porous network structures of the nanocomposite and the high charge-transfer kinetics from metallic NiS₂ nanoparticles on the highly conductive LIG scaffold (FIG. 56). Moreover, a built-in electric field is established at the interface of LIG foam and NiS₂ nanoparticles due to their different work functions (4.1 and 3.5 eV for LIG and NiS₂ measured by a Kelvin probe force microscopy), resulting in significantly improved adsorption of the polar gas molecules. Furthermore, the response of the wearable gas sensor to 10 ppm of NO₂ is much larger than that to 10 ppm of NO, CO₂, NH₃, CO, acetone, SO₂, methane, H₂S, and ethanol (FIG. 57), demonstrating excellent selectivity to NO₂. Meanwhile, various decoupling strategies can be applied to multimodal sensors for improved accuracy in practical applications.

Integration and Demonstration of the Crumpled Porous Graphene NiS₂-based Standalone Biophysical Sensing Platform

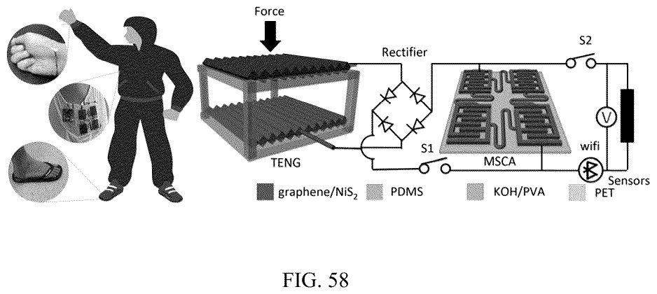

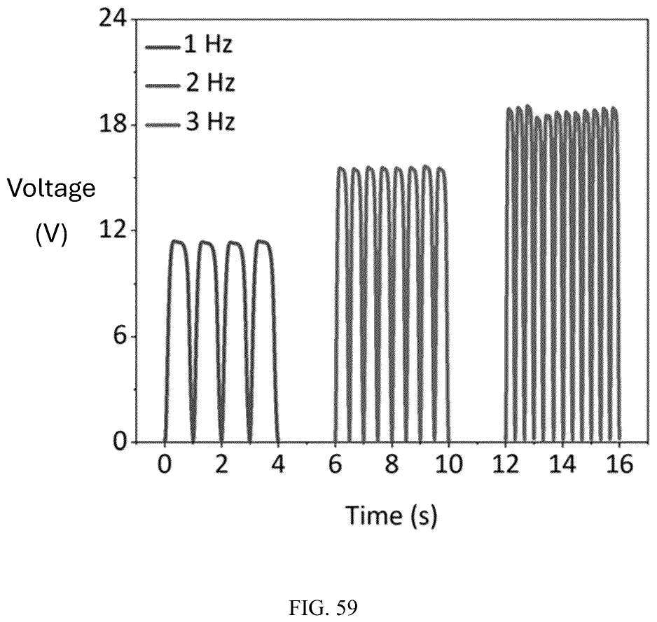

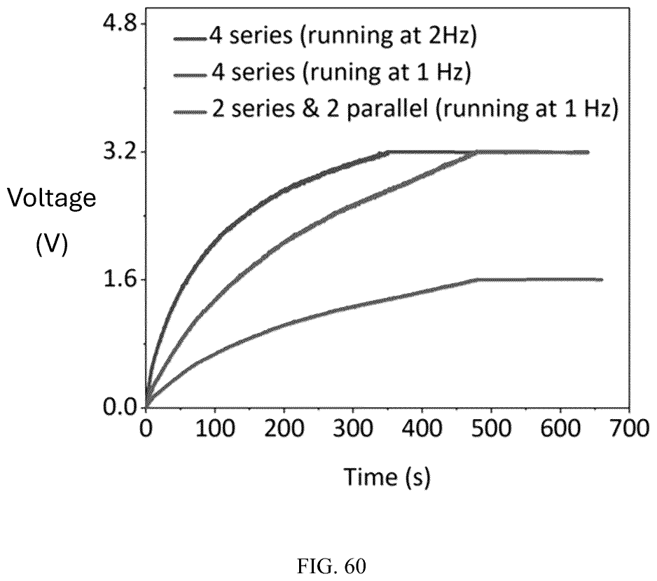

The full integration of the crumpled porous graphene/NiS₂-based wearable biophysical sensors, TENG, and MSCAs with power management circuits and wireless transmission modules (Bluetooth chips, CC2541) provides a standalone biophysical sensing platform (FIG. 58). The two TENGs attached to the shoes of a human subject (with a body weight of 80 kg) convert mechanical energy from human motion such as running at a frequency of 1, 2, or 3 Hz into electrical energy with a stable maximum output voltage of 11.4, 15.7, or 19.2 V (FIG. 59), corresponding to a peak output power density of 1.2×10⁻¹, 2.2×10⁻¹, or 3.3×10⁻¹ mW cm⁻². The harvested intermittent and alternating electrical energy from the insole TENGs is stored as electrochemical energy in the MSCAs through a bridge rectifier. As the human subject runs at a constant frequency of 1 or 2 Hz, the real-time charging voltage of the stretchable MSCAs with four MSC cells connected in series (4S, 4 cells in series) or parallel (2P*2S, 2 cells in series and then in parallel) first rapidly and then slowly increases to the stable operating voltage of 3.2 or 1.6 V after 352 or 481 s (FIG. 60). Meanwhile, the charging takes 478 s when the stretchable MSCAs consist of two MSC cells connected in series and another two connected in parallel (at 1 Hz). These results demonstrate that the self-powered charging units can efficiently harvest and store energy with an adjustable power output during running exercise.

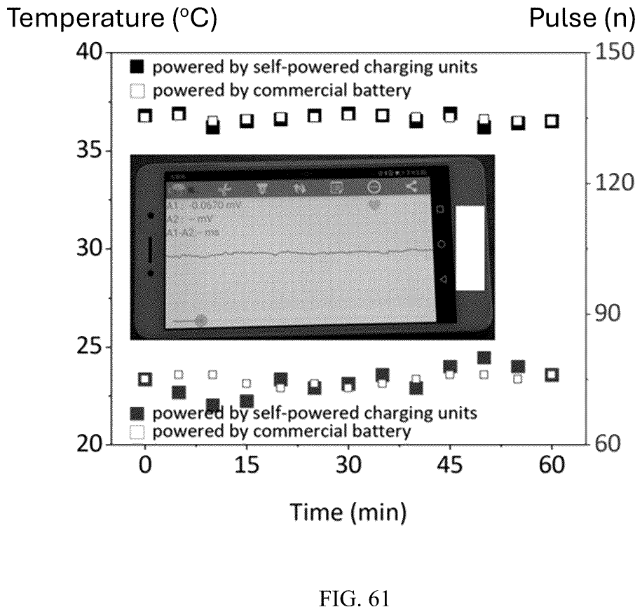

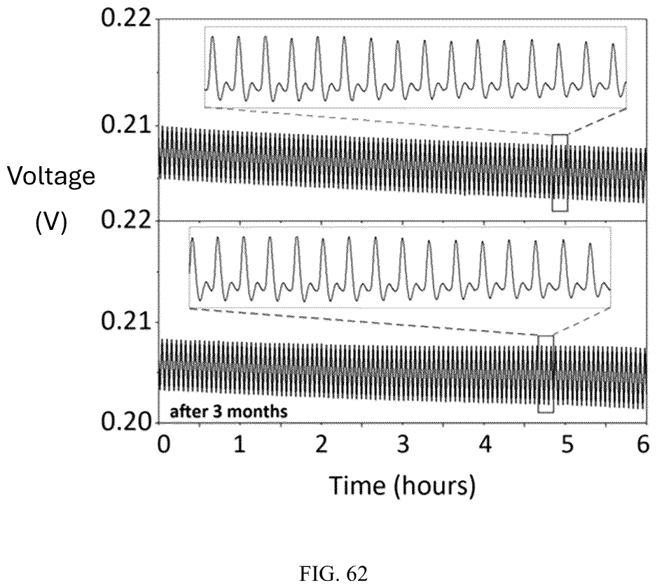

The sustained power source provided by the wearable self-powered charging units can drive low-power wearable biophysical sensors and a wireless transmission module for long-term continuous measurements. In the proof-of-the-concept demonstration, the skin temperature and pulse signals from a healthy subject running on treadmill (at 1 Hz) measured by the fully integrated standalone biophysical sensing platform are consistent with those from the integrated systems powered by a commercial battery (Nanfu, 1300 mAg, 1.5 V) during the 60-min exercise (FIG. 61). In particular, the measured skin temperature (or arterial pulse) of the healthy human subject with an average of 36.6° C. (or 74.5 bpm) and a standard deviation of 0.72 (or 0.63) from the fully integrated system agrees reasonably well with that of 36.7±0.83° C. (or 74.8±0.91 bpm) from the system powered by the commercial battery. Furthermore, the comparison in the continuously monitored radial artery pulse signal over 6 hours before (FIG. 62) and after three months (FIG. 62) indicates stable, distinguishable systolic peak (“P1”) and diastolic peak (labeled as “P2”), confirming the long-term stability of a fully integrated standalone biophysical sensing platform.

### CONCLUSIONS

In summary, we demonstrated a facile and low-cost fabrication approach for preparing 3D crumpled graphene/NiS₂ nanocomposite patterns through laser direct writing combined with electrodeposition and the pre-strain strategy. With high mechanical stretchability and electrical conductivity, the resulting functional nanomaterial composites have been demonstrated in a standalone stretchable sensing device that harvests mechanical energy with TENG and stores the converted electrical energy in MSCAs for driving various biophysical sensors. The mechanically stretchable crumpled graphene/NiS₂ nanocomposite patterns with versatile functions prepared by a facile fabrication approach hold great potential in health monitoring, personalized medicine, and human-machine interfaces.

It should be understood that modifications to the embodiments disclosed herein can be made to meet a particular set of design criteria. For instance, the number of or configuration of components or parameters may be used to meet a particular objective.

It will be apparent to those skilled in the art that numerous modifications and variations of the described examples and embodiments are possible in light of the above teachings of the disclosure. The disclosed examples and embodiments are presented for purposes of illustration only. Other alternative embodiments may include some or all of the features of the various embodiments disclosed herein. For instance, it is contemplated that a particular feature described, either individually or as part of an embodiment, can be combined with other individually described features, or parts of other embodiments. The elements and acts of the various embodiments described herein can therefore be combined to provide further embodiments.

It is the intent to cover all such modifications and alternative embodiments as may come within the true scope of this invention, which is to be given the full breadth thereof. Additionally, the disclosure of a range of values is a disclosure of every numerical value within that range, including the endpoints. Thus, while certain exemplary embodiments of the device and methods of making and using the same have been discussed and illustrated herein, it is to be distinctly understood that the invention is not limited thereto but may be otherwise variously embodied and practiced within the scope of the following claims.

What is claimed is:

1. A method of forming a crumpled nanocomposite material, the method comprising:
forming a graphene-based three-dimensional, porous scaffold by laser scribing a substrate;
depositing active nanoparticles onto the scaffold to form a nanocomposite material;
transferring the nanocomposite material onto a pre-strained elastomeric substrate;
releasing the pre-strain in the elastomeric substrate, thus forming the crumpled nanocomposite material; and
optionally removing the elastomeric substrate from the crumpled nanocomposite material.

2. The method of claim 1, wherein the active nanoparticles include transition metal sulfides represented by the formula MₓS_{y}, wherein M is a transition metal, and wherein x and y are positive integers.

3. The method of claim 2, wherein the transition metal is nickel.

4. The method of claim 3, wherein the active nanoparticles include NiS₂.

5. The method of claim 1, wherein the active nanoparticles have a diameter between 10 nm and 1000 nm.

6. The method of claim 1, wherein the active nanoparticles have a diameter between 100 nm and 600 nm.

7. The method of claim 1, wherein the amount of pre-strain applied to the elastomeric substrate is from 1% to 200%.

8. The method of claim 1, further comprising, after depositing active nanoparticles onto the scaffold to form a nanocomposite material:
annealing the nanocomposite material in an argon gas atmosphere.

9. A system comprising:
at least one sensing device, comprising:
a crumpled nanocomposite material formed from the method of claim 1,
an electrolyte layer adjacent to the crumpled nanocomposite material,
a first encapsulation layer, and
a second encapsulation layer, wherein the crumpled nanocomposite material and the electrolyte layer are positioned between the first encapsulation layer and the second encapsulation layer; and
an input/output device configured to receive data from the at least one sensing device.

10. The system of claim 9, further comprising:
a central computer device configured to receive data from the at least one sensing device and/or the input/output device.

11. The system of claim 9, wherein the data comprises one or more of motion data, temperature data, and toxic gas data.

12. The system of claim 11, wherein the at least one sensing device comprises:
a first sensing device configured to collect motion data,
a second sensing device configured to collect temperature data, and
a third sensing device configured to collect toxic gas data.

13. The system of claim 9, wherein the sensing device is configured as a triboelectric nanogenerator.

14. The system of claim 9, wherein the sensing device is configured as a micro-supercapacitor array.

15. The system of claim 9, wherein the at least one sensing device comprises:
a first sensing device configured as a triboelectric nanogenerator,
a second sensing device configured as a micro-supercapacitor array, and
a third sensing device configured as a biophysical sensor configured to collect one or more of motion data, temperature data, and toxic gas data.

16. The system of claim 15, wherein the first sensing device is configured to convert kinetic energy into electrical energy, the second sensing device is configured to store the electrical energy, wherein the electrical energy powers the third sensing device.

17. A method for collecting sensor data, the method comprising:
providing a system comprising:
at least one sensing device, comprising:
a crumpled nanocomposite material formed from the method of claim 1,
an electrolyte layer adjacent to the crumpled nanocomposite material,
a first encapsulation layer, and
a second encapsulation layer, wherein the crumpled nanocomposite material and the electrolyte layer are positioned between the first encapsulation layer and the second encapsulation layer; and
an input/output device configured to receive data from the at least one sensing device;
collecting sensor data via the at least one sensing device; and
transmitting the sensor data to an input/output device for evaluation of the sensor data.

18. The method of claim 17, wherein the sensor data comprises one or more of motion data, temperature data, and toxic gas data.

19. The method of claim 17, wherein the at least one sensing device comprises:
a first sensing device configured as a triboelectric nanogenerator,
a second sensing device configured as a micro-supercapacitor array, and
a third sensing device configured as a biophysical sensor configured to collect one or more of motion data, temperature data, and toxic gas data.

20. The method of claim 19, wherein collecting sensor data comprises:
converting kinetic energy into electrical energy via the first sensing device,
storing the electrical energy via the second sensing device,
powering the third sensing device via the electrical energy, and
collecting the sensor data via the third sensing device.
